(19) United States
(12) Patent Application Publication
(10) Pub. No.: US 2022/0110860 A1
(43) Pub. Date: Apr. 14, 2022

(54) TRANSDERMAL FORMULATIONS

(71) Applicant: NEXZOL PHARMA, INC., San Clemente, CA (US)

(72) Inventors: Joseph M. FRACASSI, San Clemente, CA (US); Thomas J. SCARLATA, San Clemente, CA (US)

(73) Assignee: NEXZOL PHARMA, INC., San Clemente, CA (US)

(21) Appl. No.: 17/560,152

(22) Filed: Dec. 22, 2021

Related U.S. Application Data
(63) Continuation of application No. PCT/US2020/038062, filed on Jun. 17, 2020.
(60) Provisional application No. 62/868,783, filed on Jun. 28, 2019.

(51) Int. Cl. A61K 9/00; A61K 45/06; A61K 47/10; A61K 47/32; A61K 47/18

(52) U.S. Cl. CPC A61K 9/0014; A61K 45/06; A61K 47/183; A61K 47/32; A61K 47/10

(57) ABSTRACT

Described herein are transdermal formulations and methods of using the same.

### CROSS-REFERENCE TO RELATED APPLICATIONS

This application claims the benefit of priority to U.S. Patent Application No. 62/868,783, filed Jun. 28, 2019, the content of which is incorporated by reference herein in its entirety.

### BACKGROUND

The present technology relates generally to the field of transdermal formulations.

### SUMMARY

In one aspect, provided herein is a transdermal formulation comprising about 0.05% w/w to about 50% w/w of an active agent and a pharmaceutically acceptable carrier, wherein the pharmaceutically acceptable carrier comprises
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w of deionized water.
   In some embodiments, the formulation does not include a phytocannabinoid.

In some embodiments, the formulation is a topical formulation. In some embodiments, the topical formulation is a semi-solid formulation selected from a gel, a lotion, a cream, an ointment, a serum, or a foam.

In some embodiments, the formulation consists of
about 0.20% w/w active agent,
about 18% w/w diethylene glycol monoethyl ether,
about 1% w/w cross-linked polyacrylic acid polymer,
about 0.3% w/w triethanolamine,
about 0.5% w/w phenoxyethanol,
about 0.05% w/w disodium EDTA dihydrate, and
q.s. deionized water.

In some embodiments, the active agent is one or more selected from anti-acne agents, anesthetics, anti-infectives, anti-rosacea agents, antibiotics, antifungals, antihistamines, anti-neoplastics, anti-psoriatics, antivirals, depigmenting agents, keratolytics, non-steroidal anti-inflammatory drugs, photochemotherapeutics, rubefacients, steroids, astringents, debriding agents, and emollients.

In some embodiments, the active agent comprises one or more anti-acne agents selected from benzoyl peroxide; tretinoin; adapalene; benzoyl peroxide and hydrocortisone; benzoyl peroxide and sulfur; resorcinol and sulfur; benzoyl peroxide and salicylic acid; benzoyl peroxide and erythromycin; benzoyl peroxide and clindamycin; erythromycin; benzoyl peroxide and adapalene; clindamycin and tretinoin; dapsone; salicylic acid; azelaic acid; clindamycin; and tetracycline.

In some embodiments, the active agent comprises one or more anesthetics selected from capsaicin, lidocaine, menthol, and methyl salicylate; pramoxine; hydrocortisone and lidocaine; tetracaine; dibucaine; prilocaine and lidocaine; menthol and lidocaine; benzalkonium chloride and lidocaine; dyclonine; phenol; camphor, methyl salicylate, and lidocaine; capsaicin, menthol, and lidocaine; cocaine; ethyl chloride; pentafluoropropane and tetrafluoroethane; pramoxine and zinc acetate; and prilocaine and lidocaine.

In some embodiments, the active agent comprises one or more anti-infectives selected from docosanol; boric acid; malathion; silver; sinecatechins; crotamiton; iodoquinol; benzyl alcohol; benzyl benzoate; cadexomer iodine; gentian violet; spinosad; ivermectin; acetic acid; imiquimod; permethrin; lindane; piperonyl butoxide and pyrethrins; hydrogen peroxide; aloe polysaccharides and iodoquinol; chloroxine; and nitrofurazone.

In some embodiments, the active agent comprises one or more anti-rosacea agents selected from azelaic acid, ivermectin, metronidazole, brimonidine, and oxymetazoline.

In some embodiments, the active agent comprises one or more antibiotics selected from mupirocin; bacitracin and polymyxin b; bacitracin, neomycin, polymyxin b, and pramoxine; gentamicin; sulfacetamide sodium; silver sulfadiazine; sulfur, retapamulin and sulfur; retapamulin; erythromycin; bacitracin, neomycin, and polymyxin b; pramoxine, neomycin, and polymyxin b; bacitracin; mafenide; neomycin and polymyxin b; neomycin; ozenoxacin; and tetracycline.

In some embodiments, the active agent comprises one or more antifungals selected from clotrimazole; tolnaftate; miconazole; clioquinol, naftifine, miconazole and zinc oxide; oxiconazole; econazole; ciclopirox; sertaconazole; ketoconazole; undecylenic acid; nystatin; efinaconazole; terbinafine; tavaborole; butenafine; ketoconazole and pyrithione zinc; luliconazole; salicylic acid and sodium thiosulfate; and sulconazole.

In some embodiments, the active agent comprises one or more antihistamines selected from diphenhydramine and doxepin.

In some embodiments, the active agent comprises one or more anti-neoplastics selected from fluorouracil, imiquimod, ingenol, and mechlorethamine.

In some embodiments, the active agent comprises one or more anti-psoriatics selected from tazarotene; betamethasone and calcipotriene; calcitriol; ammoniated mercury; anthralin; halobetasol and tazarotene; methoxsalen; and resorcinol.

In some embodiments, the active agent comprises one or more antivirals selected from penciclovir and acyclovir.

In some embodiments, the active agent comprises one or more depigmenting agents selected from fluocinolone, hydroquinone, and tretinoin; and hydroquinone.

In some embodiments, the active agent comprises one or more keratolytics selected from salicylic acid, podofilox, and Podophyllum resin.

In some embodiments, the active agent comprises one or more non-steroidal anti-inflammatory drugs selected from diclofenac; indomethacin; capsaicin and diclofenac; and ibuprofen.

In some embodiments, the active agent comprises one or more photochemotherapeutics selected from aminolevulinic acid, methoxsalen, and methyl aminolevulinate.

In some embodiments, the active agent comprises one or more rubefacients selected from trolamine salicylate; methyl salicylate; camphor and menthol and methyl salicylate; menthol; camphor and menthol; camphor; capsaicin, menthol, and methyl salicylate; camphor and phenol; capsaicin and menthol; and menthol and methyl salicylate.

In some embodiments, the active agent comprises one or more steroids selected from hydrocortisone; fluocinolone; diflorasone; prednicarbate; clocortolone; halcinonide; fluticasone; amcinonide; ammonium lactate and halobetasol; mometasone; clobetasol; flurandrenolide; desonide; betamethasone; desoximetasone; fluocinonide; halobetasol; triamcinolone; alclometasone; hydrocortisone, salicylic acid, and sulfur; and hydrocortisone and urea.

In some embodiments, the active agent comprises one or more astringents selected from witch hazel; aluminum acetate; and aluminum sulfate and calcium acetate.

In some embodiments, the active agent comprises one or more debriding agents selected from balsam peru, castor oil, and trypsin; and collagenase.

In some embodiments, the active agent comprises one or more emollients selected from urea; aloe vera; glycerin; lanolin; salicylic acid and urea; vitamins A and D; ammonium lactate; ammonium lactate and urea; hydrocortisone and urea; lactic acid and urea; petrolatum; and vitamins A, D, and E.

In some embodiments, the active agent is one or more selected from cyclobenzaprine; gabapentin; baclofen; colchicine; minoxidil; balsam peru; benzoin; dexpanthenol; diphenhydramine and hydrocortisone; lactic acid; sulfur; zinc oxide; pyrithione zinc; salicylic acid and sulfur; calamine; coal tar, salicylic acid, and sulfur; aluminum chloride hexahydrate; bimatoprost; sodium hyaluronate; coal tar; eflornithine; arnica; selenium sulfide; pimecrolimus; bentoquatam; tacrolimus; allantoin, camphor, and phenol; glycopyrronium; capsaicin; crisaborole; alitretinoi; balsam peru and castor oil; becaplermin; bexarotene; coal tar and salicylic acid; epinephrine; formaldehyde; jojoba; menthol and zinc oxide; mequinol and tretinoin; vitamin A; vitamin E; clotrimazole; dexamethasone; fluconazole; ketamine; flurbiprofen; fluticasone; or any combination thereof.

In some embodiments, the formulation includes hemp oil or a phytocannabinoid, such as cannabidiol (CBD), cannabigerol (CBG), cannabichromene (CBC), cannabinol (CBN), cannabitriol (CBT), cannabidiolic acid (CBDA), cannabigerolic acid (CBGA), cannabidivarin (CBDV), beta caryophyllene, or tetrahydrocannabinol (THC). However, in some embodiments, the formulation does not include hemp oil or a phytocannabinoid.

In some embodiments, the formulation exhibits a lag effect wherein, following four consecutive hourly applications of the formulation to skin, the amount of the active agent delivered through the skin after 21 hours is greater than the amount delivered through the skin after 5 hours, as assessed in an in vitro permeation study using human cadaver skin.

In some embodiments, the penetration enhancer comprises diethylene glycol monoethyl ether, lauryl alcohol, dimethyl sulfoxide (DMSO), dimethyl acetamide, N-methyl pyrrolidone, oleic acid, azone, oxazolidinone derivative, urea, terpene, or any combination thereof.

In some embodiments, the thickening agent comprises a cross-linked polyacrylic acid polymer; a cellulose derivative; xanthan gum, locust beam gum, guar gum or derivative thereof; alginic acid; inorganic polymer; PEMULEN™ (a copolymer of acrylic acid and C10-C30 alkyl acrylate cross-linked with allyl pentaerythritol); or any combination thereof.

In some embodiments, the buffering agent comprises triethanolamine, potassium hydroxide, cocoamidodiethyl amine, or any combination thereof.

In some embodiments, the sequestering agent comprises EDTA, or a salt and/or solvate thereof; citric acid; tartaric acid; or any combination thereof.

In some embodiments, the preservative comprises phenoxyethanol, a urea derivative, ethylhexylglycerine, hydantoin, benzoic, sorbic acid, anisic acid, or any combination thereof.

In another aspect, provided herein is a method for treating acne, bacterial skin infection, dandruff, photoaging of the skin, or rosacea, or any combination thereof, in a subject in need thereof, the method comprising topically administering a therapeutically effective amount of a transdermal formulation described herein.

In another aspect, provided herein is a method for treating allergic urticaria, anal itching, aphthous ulcer, atopic dermatitis, back pain, bacterial skin infection, external burn, cold sore, dermal ulcer, hemorrhoids, insect bites, minor cuts, minor skin irritation, muscle pain, muscle spasm, neuropathic pain, poison ivy, poison oak, poison sumac, postherpetic neuralgia, premature ejaculation, pruritus, scrapes, skin rash, sunburn, or urticaria, or any combination thereof, in a subject in need thereof, the method comprising topically administering a therapeutically effective amount of a transdermal formulation described herein.

In another aspect, provided herein is a method for treating atopic dermatitis, bacterial vaginitis, basal cell carcinoma, cold sores, Condylomata acuminata, dandruff, dermal ulcer, dermatitis, eczema, head lice, herpes simplex, human papilloma viral infection, keratosis, lichen simplex chronicus, molluscum contagiosum, pruritus, rosacea, scabies, seborrheic dermatitis, or seborrheic keratosis, or any combination thereof, in a subject in need thereof, the method comprising topically administering a therapeutically effective amount of a transdeiinal formulation described herein.

In another aspect, provided herein is a method for treating acne, bacterial vaginitis, balanoposthitis, head lice, perioral dermatitis, or rosacea, or any combination thereof, in a subject in need thereof, the method comprising topically administering a therapeutically effective amount of a transdermal formulation described herein.

In another aspect, provided herein is a method for treating acne, bacterial skin infection, external burn, dandruff, impetigo, nasal carriage of Staphylococcus aureus, paronychia, perioral dermatitis, rosacea, seborrheic dermatitis, secondary cutaneous bacterial infections, or any combination thereof, in a subject in need thereof, the method comprising topically administering a therapeutically effective amount of a transdermal formulation described herein.

In another aspect, provided herein is a method for treating androgenetic alopecia, balanoposthitis, beef tapeworm infection (Taenia saginata), cutaneous candidiasis, dandruff, diaper rash, impetigo, intertrigo, onychomycosis, fingernail onychomycosis, toenail onychomycosis, paronychia, seborrheic dermatitis, Tinea corporis, Tinea cruris, Tinea pedis, or Tinea versicolor, or any combination thereof, in a subject in need thereof, the method comprising topically administering a therapeutically effective amount of a transdermal formulation described herein.

In another aspect, provided herein is a method for treating pain, atopic dermatitis, dermatitis, eczema, lichen simplex chronicus, or pruritus, or any combination thereof, in a subject in need thereof, the method comprising topically administering a therapeutically effective amount of a transdermal formulation described herein.

In another aspect, provided herein is a method for treating Condylomata acuminata, human papilloma viral infection, keratosis, molluscum contagiosum, mycosis fungoides, skin cancer, or warts, or any combination thereof, in a subject in need thereof, the method comprising topically administering a therapeutically effective amount of a transdermal formulation described herein.

In another aspect, provided herein is a method for treating acne, bacterial skin infection, eczema, facial wrinkles, human papilloma viral infection, impetigo, psoriasis, seborrheic dermatitis, skin pigmentation disorder, or vitiligo, or any combination thereof, in a subject in need thereof, the method comprising topically administering a therapeutically effective amount of a transdermal formulation described herein.

In another aspect, provided herein is a method for treating cold sores or herpes simplex in a subject in need thereof, the method comprising topically administering a therapeutically effective amount of a transdermal formulation described herein.

In another aspect, provided herein is a method for treating melasma in a subject in need thereof, the method comprising topically administering a therapeutically effective amount of a transdermal formulation described herein.

In another aspect, provided herein is a method for treating acne, Condylomata acuminate, dandruff, human papilloma viral infection, or warts, or any combination thereof, in a subject in need thereof, the method comprising topically administering a therapeutically effective amount of a transdermal formulation described herein.

In another aspect, provided herein is a method for treating pain, keratosis, or osteoarthritis, or any combination thereof, in a subject in need thereof, the method comprising topically administering a therapeutically effective amount of a transdermal formulation described herein.

In another aspect, provided herein is a method for treating keratosis or vitiligo, or any combination thereof, in a subject in need thereof, the method comprising topically administering a therapeutically effective amount of a transdermal formulation described herein.

In another aspect, provided herein is a method for treating pain, bursitis, cold symptoms, dermatitis, osteoarthritis, pruritus, Raynaud's Syndrome, rheumatoid arthritis, or tendonitis, or any combination thereof, in a subject in need thereof, the method comprising topically administering a therapeutically effective amount of a transdermal formulation described herein.

In another aspect, provided herein is a method for treating anal itching, recurrent aphthous stomatitis, atopic dermatitis, cutaneous T-cell lymphoma, dermatitis, dermatologic lesion, eczema, granuloma annulare, hemorrhoids, intertrigo, Lichen planus, Lichen sclerosus, necrobiosis lipoidica diabeticorum, plantar fibromatosis, pruritus, psoriasis, seborrheic dermatitis, skin rash, stomatitis, or urticaria, or any combination thereof, in a subject in need thereof, the method comprising topically administering a therapeutically effective amount of a transdermal formulation described herein.

In another aspect, provided herein is a method for drying up oily skin in a subject in need thereof, the method comprising topically administering a therapeutically effective amount of a transdermal formulation described herein.

In another aspect, provided herein is a method of cleaning a wound in a subject in need thereof, the method comprising topically administering a therapeutically effective amount of a transdermal formulation described herein.

In another aspect, provided herein is a method of moisturizing skin in a subject in need thereof, the method comprising topically administering a therapeutically effective amount of a transdermal formulation described herein.

In another aspect, provided herein is a method of treating musculoskeletal pain and/or inflammation in a subject in need thereof, the method comprising, consisting essentially of, or consisting of administering to one or more regions of skin on the subject laser therapy and a transdermal formulation, wherein the transdermal formulation comprises, consists essentially of, or consists of about 0.05% w/w to about 50% w/w of a phytocannabinoid dispersed in a pharmaceutically acceptable carrier, wherein the pharmaceutically acceptable carrier comprises, consists essentially of, or consists of
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w of deionized water.

### BRIEF DESCRIPTION OF THE DRAWINGS

FIG. 1 depicts permeation results of an exemplary formulation of the present technology through human cadaver skin using Franz diffusion cells after repeat applications. Results are shown for measurements at 2, 4, 6, and 8 hour time-points.

FIG. 2 depicts permeation results of the same exemplary formulation of the present technology of FIG. 1 through human cadaver skin using Franz diffusion cells after repeat applications. Results are shown for measurements at 2, 4, 6, 8 and 24 hour time-points.

FIG. 3 depicts cannabidiol (CBD) retention results after the 24-hour time-point shown in FIG. 2.

FIG. 4 depicts permeation results of the same exemplary formulation of the present technology of FIG. 1 compared to a marketed formulation (denoted as marketed competitor) through human cadaver skin using Franz diffusion cells. Results are shown for measurements at 4, 6, 8, and 22-hour time-points.

FIG. 5 depicts CBD retention results after the 22-hour time-point shown in FIG. 4.

FIG. 6 depicts self-reported pain scores in an open label study for treatment of joint and/or muscle pain. Average pain scores were on a scale of 1 to 10.

FIG. 7 depicts delivered dose results of another exemplary formulation of the present technology compared to a marketed formulation through human cadaver skin using Franz diffusion cells. For each pair of bars, the left-hand bar represents the marketed formulation, and the right-hand bar represents the exemplary formulation of the present technology.

FIG. 8 depicts percent delivery of active agent of the exemplary formulation of the present technology of FIG. 7 compared to a marketed formulation through human cadaver skin using Franz diffusion cells. For each pair of bars, the left-hand bar represents the marketed formulation, and the right-hand bar represents the exemplary formulation of the present technology.

FIG. 9 depicts flux results of the exemplary formulation of the present technology of FIG. 7 compared to a marketed formulation through human cadaver skin using Franz diffusion cells. For each pair of bars, the left-hand bar represents the marketed formulation, and the right-hand bar represents the exemplary formulation of the present technology.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “subject” refers to an animal, such as a mammal (including a human), that has been or will be the object of treatment, observation or experiment. “Subject” and “patient” may be used interchangeably, unless otherwise indicated. Mammals include, but are not limited to, mice, rodents, rats, simians, humans, farm animals, dogs, cats, sport animals, and pets. The methods described herein may be useful in human therapy and/or veterinary applications. In some embodiments, the subject is a mammal. In some embodiments, the subject is a human.

The terms “therapeutically effective amount” and “effective amount” are used interchangeably and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease or disorder, or the manner of administration as determined by a qualified prescriber or caregiver.

The term “treatment” or “treating” means administering a formulation disclosed herein for the purpose of: (i) delaying the onset of a disease/disorder, that is, causing the clinical symptoms of the disease/disorder not to develop or delaying the development thereof; (ii) inhibiting the disease/disorder, that is, arresting the development of clinical symptoms; and/or (iii) relieving the disease/disorder, that is, causing the regression of clinical symptoms or the severity thereof.

By “pharmaceutically acceptable” is meant a material that is not biologically or otherwise undesirable, e.g., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. A pharmaceutically acceptable salt of an active agent can be used instead of the free base form of the active agent in any formulation disclosed herein.

As used herein, the term “musculoskeletal” refers to joints, tendons, ligaments, skeletal muscles (e.g., muscles that contract to pull on tendons and move the bones of the skeleton, maintain posture and body position, support soft tissues, guard entrances and exits to the digestive and urinary tracts; and maintain body temperature), nerves, and cartilage. Accordingly, in some embodiments, musculoskeletal pain/inflammation is located at one or more joints, tendons, ligaments, skeletal muscles, nerves, and cartilage.

As used herein, the term “transdermal” refers to topical application to a skin surface for local and/or systemic effect(s) depending on the active agent in the formulation.

Unless defined otherwise, all technical and scientific terms used herein have the same meaning as commonly understood by one of ordinary skill in the art to which this present invention belongs. Although any methods and materials similar or equivalent to those described herein can also be used in the practice or testing of the present invention, representative illustrative methods and materials are described herein.

Transdermal Formulations

In one aspect, provided herein are transdermal formulations comprising, consisting essentially of, or consisting of about 0.05% w/w to about 50% w/w active agent and pharmaceutically acceptable excipients.

In another aspect, provided herein are transdermal formulations comprising, consisting essentially of, or consisting of about 0.05% w/w to about 50% w/w active agent and a pharmaceutically acceptable carrier comprising, consisting essentially of, or consisting of:
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w deionized water.

In another aspect, provided herein are transdermal formulations comprising, consisting essentially of, or consisting of about 0.05% w/w to about 30% w/w active agent and a pharmaceutically acceptable carrier comprising, consisting essentially of, or consisting of
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w deionized water.

In another aspect, provided herein are transdermal formulations comprising, consisting essentially of, or consisting of about 0.05% w/w to about 50% w/w active agent dispersed in a pharmaceutically acceptable carrier comprising, consisting essentially of, or consisting of:
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w deionized water.

In another aspect, provided herein are transdermal formulations comprising, consisting essentially of, or consisting of about 0.05% w/w to about 30% w/w active agent dispersed in a pharmaceutically acceptable carrier comprising, consisting essentially of, or consisting of
about 3% w/w to about 30% w/w penetration enhancer,
about 0.8% w/w to about 1.3% w/w thickening agent,
about 0.25% w/w to about 6% why buffering agent,
about 0.05% w/w to about 0.08% w/w sequestering agent,
about 0.4% w/w to about 0.8% w/w preservative, and
up to about 95.45% w/w deionized water.

In another aspect, provided herein are transdermal formulations consisting of
about 0.2% w/w active agent,
about 18% w/w diethylene glycol monoethyl ether,
about 1% w/w cross-linked polyacrylic acid polymer,
about 0.3% w/w triethanolamine,
about 0.5% w/w phenoxyethanol,
about 0.05% w/w disodium EDTA dihydrate, and
q.s. deionized water.

In any embodiments, the formulation disclosed herein may be in the form of a topical formulation. Topical formulations include, but are not limited to, gels, lotions, creams, ointments, pastes, serums, foams, sprays, powders, or liquids (e.g., suspension or solution). In any embodiments, the topical formulation may be a semi-solid formulation. A semi-solid formulation includes, but is not limited to, a gel, a lotion, a cream, an ointment, a suspension, a paste, a serum, and a foam.

In any embodiments, the formulation disclosed herein may be in the form of a lotion, cream, gel, paste, serum, or ointment. In some embodiments, the formulation disclosed herein is a gel.

Active Agent

The active agent may be selected from one or more of anti-acne agents, anesthetics, anti-infectives, anti-rosacea agents, antibiotics, antifungals, antihistamines, anti-neoplastics, anti-psoriatics, antivirals, depigmenting agents, keratolytics, non-steroidal anti-inflammatory drugs, photochemotherapeutics, rubefacients, steroids, astringents, debriding agents, and emollients.

Anti-acne agents may be selected from one or more of benzoyl peroxide; tretinoin; adapalene; benzoyl peroxide and hydrocortisone; benzoyl peroxide and sulfur; resorcinol and sulfur; benzoyl peroxide and salicylic acid; benzoyl peroxide and erythromycin; benzoyl peroxide and clindamycin; erythromycin; benzoyl peroxide and adapalene; clindamycin and tretinoin; dapsone; salicylic acid; azelaic acid; clindamycin; and tetracycline.

In some embodiments, the active agent is an anti-acne agent, and the anti-acne agent is benzoyl peroxide. In some embodiments, the active agent is an anti-acne agent, and the anti-acne agent is tretinoin. In some embodiments, the active agent is an anti-acne agent, and the anti-acne agent is adapalene. In some embodiments, the active agent is an anti-acne agent, and the anti-acne agent is benzoyl peroxide and hydrocortisone. In some embodiments, the active agent is an anti-acne agent, and the anti-acne agent is benzoyl peroxide and sulfur. In some embodiments, the active agent is an anti-acne agent, and the anti-acne agent is resorcinol and sulfur. In some embodiments, the active agent is an anti-acne agent, and the anti-acne agent is benzoyl peroxide and salicylic acid. In some embodiments, the active agent is an anti-acne agent, and the anti-acne agent is benzoyl peroxide and erythromycin. In some embodiments, the active agent is an anti-acne agent, and the anti-acne agent is benzoyl peroxide and clindamycin. In some embodiments, the active agent is an anti-acne agent, and the anti-acne agent is erythromycin. In some embodiments, the active agent is an anti-acne agent, and the anti-acne agent is benzoyl peroxide and adapalene. In some embodiments, the active agent is an anti-acne agent, and the anti-acne agent is clindamycin and tretinoin. In some embodiments, the active agent is an anti-acne agent, and the anti-acne agent is dapsone. In some embodiments, the active agent is an anti-acne agent, and the anti-acne agent is salicylic acid. In some embodiments, the active agent is an anti-acne agent, and the anti-acne agent is azelaic acid. In some embodiments, the active agent is an anti-acne agent, and the anti-acne agent is clindamycin. In some embodiments, the active agent is an anti-acne agent, and the anti-acne agent is tetracycline.

Anesthetics may be selected from one or more of capsaicin, lidocaine, menthol, and methyl salicylate; pramoxine; hydrocortisone and lidocaine; tetracaine; dibucaine; prilocaine and lidocaine; menthol and lidocaine; benzalkonium chloride and lidocaine; dyclonine; phenol; camphor, methyl salicylate, and lidocaine; capsaicin, menthol, and lidocaine; cocaine; ethyl chloride; pentafluoropropane and tetrafluoroethane; pramoxine and zinc acetate; and prilocaine and lidocaine.

In some embodiments, the active agent is an anesthetic, and the anesthetic is capsaicin, lidocaine, menthol, and methyl salicylate. In some embodiments, the active agent is an anesthetic, and the anesthetic is pramoxine. In some embodiments, the active agent is an anesthetic, and the anesthetic is hydrocortisone and lidocaine. In some embodiments, the active agent is an anesthetic, and the anesthetic is tetracaine. In some embodiments, the active agent is an anesthetic, and the anesthetic is dibucaine. In some embodiments, the active agent is an anesthetic, and the anesthetic is prilocaine and lidocaine. In some embodiments, the active agent is an anesthetic, and the anesthetic is menthol and lidocaine. In some embodiments, the active agent is an anesthetic, and the anesthetic is benzalkonium chloride and lidocaine. In some embodiments, the active agent is an anesthetic, and the anesthetic is dyclonine. In some embodiments, the active agent is an anesthetic, and the anesthetic is phenol. In some embodiments, the active agent is an anesthetic, and the anesthetic is camphor, methyl salicylate, and lidocaine. In some embodiments, the active agent is an anesthetic, and the anesthetic is capsaicin, menthol, and lidocaine. In some embodiments, the active agent is an anesthetic, and the anesthetic is cocaine. In some embodiments, the active agent is an anesthetic, and the anesthetic is ethyl chloride. In some embodiments, the active agent is an anesthetic, and the anesthetic is pentafluoropropane and tetrafluoroethane. In some embodiments, the active agent is an anesthetic, and the anesthetic is pramoxine and zinc acetate. In some embodiments, the active agent is an anesthetic, and the anesthetic is prilocaine and lidocaine.

Anti-infectives may be selected from one or more of docosanol; boric acid; malathion; silver; sinecatechins; crotamiton; iodoquinol; benzyl alcohol; benzyl benzoate; cadexomer iodine; gentian violet; spinosad; ivermectin; acetic acid; imiquimod; permethrin; lindane; piperonyl butoxide and pyrethrins; hydrogen peroxide; aloe polysaccharides and iodoquinol; chloroxine; and nitrofurazone.

In some embodiments, the active agent is an anti-infective, and the anti-infective is docosanol. In some embodiments, the active agent is an anti-infective, and the anti-infective is boric acid. In some embodiments, the active agent is an anti-infective, and the anti-infective is malathion. In some embodiments, the active agent is an anti-infective, and the anti-infective is silver. In some embodiments, the active agent is an anti-infective, and the anti-infective is sinecatechins. In some embodiments, the active agent is an anti-infective, and the anti-infective is crotamiton. In some embodiments, the active agent is an anti-infective, and the anti-infective is iodoquinol. In some embodiments, the active agent is an anti-infective, and the anti-infective is benzyl alcohol. In some embodiments, the active agent is an anti-infective, and the anti-infective is benzyl benzoate. In some embodiments, the active agent is an anti-infective, and the anti-infective is cadexomer iodine. In some embodiments, the active agent is an anti-infective, and the anti-infective is gentian violet. In some embodiments, the active agent is an anti-infective, and the anti-infective is spinosad. In some embodiments, the active agent is an anti-infective, and the anti-infective is ivermectin. In some embodiments, the active agent is an anti-infective, and the anti-infective is acetic acid. In some embodiments, the active agent is an anti-infective, and the anti-infective is imiquimod. In some embodiments, the active agent is an anti-infective, and the anti-infective is permethrin. In some embodiments, the active agent is an anti-infective, and the anti-infective is lindane. In some embodiments, the active agent is an anti-infective, and the anti-infective is piperonyl butoxide and pyrethrins. In some embodiments, the active agent is an anti-infective, and the anti-infective is hydrogen peroxide. In some embodiments, the active agent is an anti-infective, and the anti-infective is aloe polysaccharides and iodoquinol. In some embodiments, the active agent is an anti-infective, and the anti-infective is chloroxine. In some embodiments, the active agent is an anti-infective, and the anti-infective is nitrofurazone.

Anti-rosacea agents may be selected from one or more of azelaic acid, ivermectin, metronidazole, brimonidine, and oxymetazoline.

In some embodiments, the active agent is an anti-rosacea agent, and the anti-rosacea agent is azelaic acid. In some embodiments, the active agent is an anti-rosacea agent, and the anti-rosacea agent is ivermectin. In some embodiments, the active agent is an anti-rosacea agent, and the anti-rosacea agent is metronidazole. In some embodiments, the active agent is an anti-rosacea agent, and the anti-rosacea agent is brimonidine. In some embodiments, the active agent is an anti-rosacea agent, and the anti-rosacea agent is oxymetazoline.

Antibiotics may be selected from one or more of mupirocin; bacitracin and polymyxin b; bacitracin, neomycin, polymyxin b, and pramoxine; gentamicin; sulfacetamide sodium; silver sulfadiazine; sulfur, retapamulin and sulfur; retapamulin; erythromycin; bacitracin, neomycin, and polymyxin b; pramoxine, neomycin, and polymyxin b; bacitracin; mafenide; neomycin and polymyxin b; neomycin; ozenoxacin; and tetracycline.

In some embodiments, the active agent is an antibiotic, and the antibiotic is mupirocin. In some embodiments, the active agent is an antibiotic, and the antibiotic is bacitracin and polymyxin b. In some embodiments, the active agent is an antibiotic, and the antibiotic is bacitracin, neomycin, polymyxin b, and pramoxine. In some embodiments, the active agent is an antibiotic, and the antibiotic is gentamicine. In some embodiments, the active agent is an antibiotic, and the antibiotic is sulfacetamide sodium. In some embodiments, the active agent is an antibiotic, and the antibiotic is silver sulfadiazine. In some embodiments, the active agent is an antibiotic, and the antibiotic is sulfur, retapamulin and sulfur. In some embodiments, the active agent is an antibiotic, and the antibiotic is retapamulin. In some embodiments, the active agent is an antibiotic, and the antibiotic is erythromycin. In some embodiments, the active agent is an antibiotic, and the antibiotic is bacitracin, neomycin, and polymyxin b. In some embodiments, the active agent is an antibiotic, and the antibiotic is pramoxine, neomycin, and polymyxin b. In some embodiments, the active agent is an antibiotic, and the antibiotic is bacitracin. In some embodiments, the active agent is an antibiotic, and the antibiotic is mafenide. In some embodiments, the active agent is an antibiotic, and the antibiotic is neomycin and polymyxin b. In some embodiments, the active agent is an antibiotic, and the antibiotic is neomycin. In some embodiments, the active agent is an antibiotic, and the antibiotic is ozenoxacin. In some embodiments, the active agent is an antibiotic, and the antibiotic is tetracycline.

Antifungals may be selected from one or more of clotrimazole; tolnaftate; miconazole; clioquinol, naftifine, miconazole and zinc oxide; oxiconazole; econazole; ciclopirox; sertaconazole; ketoconazole; undecylenic acid; nystatin; efinaconazole; terbinafine; tavaborole; butenafine; ketoconazole and pyrithione zinc; luliconazole; salicylic acid and sodium thiosulfate; and sulconazole.

In some embodiments, the active agent is an antifungal, and the antifungal is clotrimazole. In some embodiments, the active agent is an antifungal, and the antifungal is tolnaftate. In some embodiments, the active agent is an antifungal, and the antifungal is miconazole. In some embodiments, the active agent is an antifungal, and the antifungal is clioquinol, naftifine, miconazole and zinc oxide. In some embodiments, the active agent is an antifungal, and the antifungal is oxiconazole. In some embodiments, the active agent is an antifungal, and the antifungal is econazole. In some embodiments, the active agent is an antifungal, and the antifungal is ciclopirox. In some embodiments, the active agent is an antifungal, and the antifungal is sertaconazole. In some embodiments, the active agent is an antifungal, and the antifungal is ketoconazole. In some embodiments, the active agent is an antifungal, and the antifungal is undecylenic acid. In some embodiments, the active agent is an antifungal, and the antifungal is nystatin. In some embodiments, the active agent is an antifungal, and the antifungal is efinaconazole. In some embodiments, the active agent is an antifungal, and the antifungal is terbinafine. In some embodiments, the active agent is an antifungal, and the antifungal is tavaborole. In some embodiments, the active agent is an antifungal, and the antifungal is butenafine. In some embodiments, the active agent is an antifungal, and the antifungal is ketoconazole and pyrithione zinc. In some embodiments, the active agent is an antifungal, and the antifungal is luliconazole. In some embodiments, the active agent is an antifungal, and the antifungal is salicylic acid and sodium thiosulfate. In some embodiments, the active agent is an antifungal, and the antifungal is sulconazole.

Antihistamines may be selected from diphenhydramine and doxepin, or a combination thereof.

In some embodiments, the active agent is an antihistamine, and the antihistamine is diphenhydramine. In some embodiments, the active agent is an antihistamine, and the antihistamine is doxepin.

Anti-neoplastics may be selected from one or more of fluorouracil, imiquimod, ingenol, and mechlorethamine.

In some embodiments, the active agent is an anti-neoplastic, and the anti-neoplastic is fluorouracil. In some embodiments, the active agent is an anti-neoplastic, and the anti-neoplastic is imiquimod. In some embodiments, the active agent is an anti-neoplastic, and the anti-neoplastic is ingenol. In some embodiments, the active agent is an anti-neoplastic, and the anti-neoplastic is mechlorethamine.

Anti-psoriatics may be selected from one or more of tazarotene; betamethasone and calcipotriene; calcitriol; ammoniated mercury; anthralin; halobetasol and tazarotene; methoxsalen; and resorcinol.

In some embodiments, the active agent is an anti-psoriatic, and the anti-psoriatic is tazarotene. In some embodiments, the active agent is an anti-psoriatic, and the anti-psoriatic is betamethasone and calcipotriene. In some embodiments, the active agent is an anti-psoriatic, and the anti-psoriatic is calcitriol. In some embodiments, the active agent is an anti-psoriatic, and the anti-psoriatic is ammoniated mercury. In some embodiments, the active agent is an anti-psoriatic, and the anti-psoriatic is anthralin. In some embodiments, the active agent is an anti-psoriatic, and the anti-psoriatic is halobetasol and tazarotene. In some embodiments, the active agent is an anti-psoriatic, and the anti-psoriatic is methoxsalen. In some embodiments, the active agent is an anti-psoriatic, and the anti-psoriatic is resorcinol.

Antivirals may be selected from penciclovir and acyclovir, or a combination thereof.

In some embodiments, the active agent is an antiviral, and the antiviral is penciclovir. In some embodiments, the active agent is an antiviral, and the antiviral is acyclovir.

Depigmenting agents may be selected from fluocinolone, hydroquinone, and tretinoin; and hydroquinone, or a combination thereof.

In some embodiments, the active agent is a depigmenting agent, and the depigmenting agent is fluocinolone, hydroquinone, and tretinoin. In some embodiments, the active agent is a depigmenting agent, and the depigmenting agent is hydroquinone.

Keratolytics may be selected from one or more of salicylic acid, podofilox, and Podophyllum resin.

In some embodiments, the active agent is a keratolytic, and the keratolytic is salicylic acid. In some embodiments, the active agent is a keratolytic, and the keratolytic is podofilox. In some embodiments, the active agent is a keratolytic, and the keratolytic is Podophyllum resin.

Non-steroidal anti-inflammatory drugs may be selected from one or more of diclofenac; indomethacin; capsaicin and diclofenac; and ibuprofen.

In some embodiments, the active agent is a non-steroidal anti-inflammatory drug, and the non-steroidal anti-inflammatory drug is diclofenac. In some embodiments, the active agent is a non-steroidal anti-inflammatory drug, and the non-steroidal anti-inflammatory drug is indomethacin. In some embodiments, the active agent is a non-steroidal anti-inflammatory drug, and the non-steroidal anti-inflammatory drug is capsaicin and diclofenac. In some embodiments, the active agent is a non-steroidal anti-inflammatory drug, and the non-steroidal anti-inflammatory drug is ibuprofen.

Photochemotherapeutics may be selected from one or more of aminolevulinic acid, methoxsalen, and methyl aminolevulinate.

In some embodiments, the active agent is a photochemotherapeutic, and the photochemotherapeutic is aminolevulinic acid. In some embodiments, the active agent is a photochemotherapeutic, and the photochemotherapeutic is methoxsalen. In some embodiments, the active agent is a photochemotherapeutic, and the photochemotherapeutic is methyl aminolevulinate.

Rubefacients may be selected from one or more of trolamine salicylate; methyl salicylate; camphor, menthol, and methyl salicylate; menthol; camphor and menthol; camphor; capsaicin, menthol, and methyl salicylate; camphor and phenol; capsaicin and menthol; and menthol and methyl salicylate.

In some embodiments, the active agent is a rubefacient, and the rubefacient is trolamine salicylate. In some embodiments, the active agent is a rubefacient, and the rubefacient is methyl salicylate. In some embodiments, the active agent is a rubefacient, and the rubefacient is camphor, menthol, and methyl salicylate. In some embodiments, the active agent is a rubefacient, and the rubefacient is menthol. In some embodiments, the active agent is a rubefacient, and the rubefacient is camphor and menthol. In some embodiments, the active agent is a rubefacient, and the rubefacient is camphor. In some embodiments, the active agent is a rubefacient, and the rubefacient is capsaicin, menthol, and methyl salicylate. In some embodiments, the active agent is a rubefacient, and the rubefacient is camphor and phenol. In some embodiments, the active agent is a rubefacient, and the rubefacient is capsaicin and menthol. In some embodiments, the active agent is a rubefacient, and the rubefacient is menthol and methyl salicylate.

Steroids may be selected from one or more of hydrocortisone; fluocinolone; diflorasone; prednicarbate; clocortolone; halcinonide; fluticasone; amcinonide; ammonium lactate and halobetasol; mometasone; clobetasol; flurandrenolide; desonide; betamethasone; desoximetasone; fluocinonide; halobetasol; triamcinolone; alclometasone; hydrocortisone, salicylic acid, and sulfur; and hydrocortisone and urea.

In some embodiments, the active agent is a steroid, and the steroid is hydrocortisone. In some embodiments, the active agent is a steroid, and the steroid is fluocinolone. In some embodiments, the active agent is a steroid, and the steroid is diflorasone. In some embodiments, the active agent is a steroid, and the steroid is prednicarbate. In some embodiments, the active agent is a steroid, and the steroid is clocortolone. In some embodiments, the active agent is a steroid, and the steroid is halcinonide. In some embodiments, the active agent is a steroid, and the steroid is fluticasone. In some embodiments, the active agent is a steroid, and the steroid is amcinonide. In some embodiments, the active agent is a steroid, and the steroid is ammonium lactate and halobetasol. In some embodiments, the active agent is a steroid, and the steroid is mometasone. In some embodiments, the active agent is a steroid, and the steroid is clobetasol. In some embodiments, the active agent is a steroid, and the steroid is flurandrenolide. In some embodiments, the active agent is a steroid, and the steroid is desonide. In some embodiments, the active agent is a steroid, and the steroid is betamethasone. In some embodiments, the active agent is a steroid, and the steroid is desoximetasone. In some embodiments, the active agent is a steroid, and the steroid is fluocinonide. In some embodiments, the active agent is a steroid, and the steroid is halobetasol. In some embodiments, the active agent is a steroid, and the steroid is triamcinolone. In some embodiments, the active agent is a steroid, and the steroid is alclometasone. In some embodiments, the active agent is a steroid, and the steroid is hydrocortisone, salicylic acid, and sulfur. In some embodiments, the active agent is a steroid, and the steroid is hydrocortisone and urea.

Astringents may be selected from one or more of witch hazel; aluminum acetate; and aluminum sulfate and calcium acetate.

In some embodiments, the active agent is an astringent, and the astringent is witch hazel. In some embodiments, the active agent is an astringent, and the astringent is aluminum acetate. In some embodiments, the active agent is an astringent, and the astringent is aluminum sulfate and calcium acetate.

Debriding agents may be selected from one or more of balsam peru, castor oil, and trypsin; and collagenase.

In some embodiments, the active agent is a debriding agent, and the debriding agent is balsam peru, castor oil, and trypsin. In some embodiments, the active agent is a debriding agent, and the debriding agent is collagenase.

Emollients may be selected from one or more of urea; aloe vera; glycerin; lanolin; salicylic acid and urea; vitamins A and D; ammonium lactate; ammonium lactate and urea; hydrocortisone and urea; lactic acid and urea; petrolatum; and vitamins A, D, and E.

In some embodiments, the active agent is an emollient, and the emollient is urea. In some embodiments, the active agent is an emollient, and the emollient is aloe vera. In some embodiments, the active agent is an emollient, and the emollient is glycerin. In some embodiments, the active agent is an emollient, and the emollient is lanolin. In some embodiments, the active agent is an emollient, and the emollient is salicylic acid and urea. In some embodiments, the active agent is an emollient, and the emollient is vitamins A and D. In some embodiments, the active agent is an emollient, and the emollient is ammonium lactate. In some embodiments, the active agent is an emollient, and the emollient is ammonium lactate and urea. In some embodiments, the active agent is an emollient, and the emollient is hydrocortisone and urea. In some embodiments, the active agent is an emollient, and the emollient is lactic acid and urea. In some embodiments, the active agent is an emollient, and the emollient is petrolatum. In some embodiments, the active agent is an emollient, and the emollient is vitamins A, D, and E.

The active agent may be one or more selected from cyclobenzaprine; gabapentin; baclofen; colchicine; minoxidil; balsam peru; benzoin; dexpanthenol; diphenhydramine and hydrocortisone; lactic acid; sulfur; zinc oxide; pyrithione zinc; salicylic acid and sulfur; calamine; coal tar, salicylic acid, and sulfur; aluminum chloride hexahydrate; bimatoprost; sodium hyaluronate; coal tar; eflornithine; arnica; selenium sulfide; pimecrolimus; bentoquatam; tacrolimus; allantoin, camphor, and phenol; glycopyrronium; capsaicin; crisaborole; alitretinoi; balsam peru and castor oil; becaplermin; bexarotene; coal tar and salicylic acid; epinephrine; formaldehyde; jojoba; menthol and zinc oxide; mequinol and tretinoin; vitamin A; vitamin E; clotrimazole; dexamethasone; fluconazole; ketamine; flurbiprofen; fluticasone; or any combination thereof.

In some embodiments, the active agent comprises hemp oil or a phytocannabinoid, such as cannabidiol (CBD), cannabigerol (CBG), cannabichromene (CBC), cannabinol (CBN), cannabitriol (CBT), cannabidiolic acid (CBDA), cannabigerolic acid (CBGA), cannabidivarin (CBDV), beta caryophyllene, or tetrahydrocannabinol (THC). However, in some embodiments, the formulation does not include hemp oil, does not include a phytocannabinoid, or does not include hemp oil and does not include a phytocannabinoid.

As used herein, a “phytocannabinoid” may be synthetic or natural. As used herein, a “natural phytocannabinoid” refers to a phytocannabinoid isolated from a natural source, such as a plant. As used herein, a “synthetic phytocannabinoid” refers to a phytocannabinoid prepared synthetically. In some embodiments, the natural phytocannibinoid is a hemp-derived phytocannabinoid. In some embodiments, hemp oil comprises one or more phytocannabinoids.

In any of these embodiments, the active agent may be microencapsulated. In some embodiments, the microencapsulated active agent comprises, consists essentially of, or consists of the active agent encapsulated within liposomes. In some embodiments, the active agent is not microencapsulated.

In any embodiments, the active agent may be present in the formulation disclosed herein in an amount of about 0.05% w/w to about 50% w/w. This includes about 0.05% w/w to about 45% w/w, about 0.05% w/w to about 40% w/w, about 0.05% w/w to about 35% w/w, about 0.05% w/w to about 30% w/w, about 0.05% w/w to about 25% w/w, about 0.05% w/w to about 20% w/w, about 0.05% w/w to about 15% w/w, about 0.05% w/w to about 10% w/w, about 0.05% w/w to about 5% w/w, about 0.05% w/w to about 4% w/w, about 0.05% w/w to about 3% w/w, about 0.05% w/w to about 2% w/w, about 0.05% w/w to about 1% w/w, about 0.05% w/w to about 0.5% w/w, about 0.1% w/w to about 45% w/w, about 0.1% w/w to about 40% w/w, about 0.1% w/w to about 35% w/w, about 0.1% w/w to about 30% w/w, about 0.1% w/w to about 25% w/w, about 0.1% w/w to about 20% w/w, about 0.1% w/w to about 15% w/w, about 0.1% w/w to about 10% w/w, about 0.1% w/w to about 5% w/w, about 0.1% w/w to about 4% w/w, about 0.1% w/w to about 3% w/w, about 0.1% w/w to about 2% w/w, about 0.1% w/w to about 1% w/w, about 0.1% w/w to about 0.5% w/w, about 1% w/w to about 45% w/w, about 1% w/w to about 40% w/w, about 1% w/w to about 35% w/w, about 1% w/w to about 30% w/w, about 1% w/w to about 25% w/w, about 1% w/w to about 20% w/w, about 1% w/w to about 15% w/w, about 1% w/w to about 10% w/w, about 1% w/w to about 5% w/w, about 5% w/w to about 45% w/w, about 5% w/w to about 40% w/w, about 5% w/w to about 35% w/w, about 5% w/w to about 30% w/w, about 5% w/w to about 25% w/w, about 5% w/w to about 20% w/w, about 5% w/w to about 15% w/w, about 5% w/w to about 10% w/w, 10% w/w to about 50% w/w, 10% w/w to about 45% w/w, about 10% w/w to about 40% w/w, about 10% w/w to about 35% w/w, about 10% w/w to about 30% w/w, about 10% w/w to about 25% w/w, about 10% w/w to about 20% w/w, and about 10% w/w to about 15% w/w. Thus, the active agent may be present in the formulation disclosed herein in an amount of about 0.05, 0.06, 0.07, 0.08, 0.09, 0.10, 0.20, 0.30, 0.40, 0.50, 0.60, 0.70, 0.80, 0.90, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% w/w, including increments therein.

In any embodiments, the active agent may be present in the formulation disclosed herein in an amount of about 1 mg to about 500 mg. This includes about 1 mg to about 25 mg; about 1 mg to about 50 mg; about 1 mg to about 75 mg; about 1 mg to about 100 mg, 1 mg to about 125 mg, 1 mg to about 150 mg, 1 mg to about 175 mg, 1 mg to about 200 mg, 1 mg to about 225 mg, 1 mg to about 250 mg, 1 mg to about 275 mg, 1 mg to about 300 mg, 1 mg to about 325 mg, 1 mg to about 350 mg, 1 mg to about 375 mg, 1 mg to about 400 mg, 1 mg to about 425 mg, 1 mg to about 450 mg, 1 mg to about 475 mg; about 25 mg to about 50 mg, about 25 mg to about 75 mg, about 25 mg to about 100 mg, 25 mg to about 125 mg, 25 mg to about 150 mg, 25 mg to about 175 mg, 25 mg to about 200 mg, 25 mg to about 225 mg, 25 mg to about 250 mg, 25 mg to about 275 mg, 25 mg to about 300 mg, 25 mg to about 325 mg, 25 mg to about 350 mg, 25 mg to about 375 mg, 25 mg to about 400 mg, 25 mg to about 425 mg, 25 mg to about 450 mg, 25 mg to about 475 mg; about 50 mg to about 100 mg, 50 mg to about 125 mg, 50 mg to about 150 mg, 50 mg to about 175 mg, 50 mg to about 200 mg, 50 mg to about 225 mg, 50 mg to about 250 mg, 50 mg to about 275 mg, 50 mg to about 300 mg, 50 mg to about 325 mg, 50 mg to about 350 mg, 50 mg to about 375 mg, 50 mg to about 400 mg, 50 mg to about 425 mg, 50 mg to about 450 mg, 50 mg to about 475 mg; 100 mg to about 125 mg, 100 mg to about 150 mg, 100 mg to about 175 mg, 100 mg to about 200 mg, 100 mg to about 225 mg, 100 mg to about 250 mg, 100 mg to about 275 mg, 100 mg to about 300 mg, 100 mg to about 325 mg, 100 mg to about 350 mg, 100 mg to about 375 mg, 100 mg to about 400 mg, 100 mg to about 425 mg, 100 mg to about 450 mg, 100 mg to about 475 mg, and 100 mg to about 500 mg. Thus, the active agent may be present in the formulation disclosed herein in an amount of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg, including increments therein.

In any embodiments, the active agent may be present in the formulation disclosed herein in an amount of about 1 mg/mL to about 500 mg/mL. This includes about 1 mg/mL to about 25 mg/mL; about 1 mg/mL to about 50 mg/mL; about 1 mg/mL to about 75 mg/mL; about 1 mg/mL to about 100 mg/mL, 1 mg/mL to about 125 mg/mL, 1 mg/mL to about 150 mg/mL, 1 mg/mL to about 175 mg/mL, 1 mg/mL to about 200 mg/mL, 1 mg/mL to about 225 mg/mL, 1 mg/mL to about 250 mg/mL, 1 mg/mL to about 275 mg/mL, 1 mg/mL to about 300 mg/mL, 1 mg/mL to about 325 mg/mL, 1 mg/mL to about 350 mg/mL, 1 mg/mL to about 375 mg/mL, 1 mg/mL to about 400 mg/mL, 1 mg/mL to about 425 mg/mL, 1 mg/mL to about 450 mg/mL, 1 mg/mL to about 475 mg/mL; about 25 mg/mL to about 50 mg/mL, about 25 mg/mL to about 75 mg/mL, about 25 mg/mL to about 100 mg/mL, 25 mg/mL to about 125 mg/mL, 25 mg/mL to about 150 mg/mL, 25 mg/mL to about 175 mg/mL, 25 mg/mL to about 200 mg/mL, 25 mg/mL to about 225 mg/mL, 25 mg/mL to about 250 mg/mL, 25 mg/mL to about 275 mg/mL, 25 mg/mL to about 300 mg/mL, 25 mg/mL to about 325 mg/mL, 25 mg/mL to about 350 mg/mL, 25 mg/mL to about 375 mg/mL, 25 mg/mL to about 400 mg/mL, 25 mg/mL to about 425 mg/mL, 25 mg/mL to about 450 mg/mL, 25 mg/mL to about 475 mg/mL; about 50 mg/mL to about 100 mg/mL, 50 mg/mL to about 125 mg/mL, 50 mg/mL to about 150 mg/mL, 50 mg/mL to about 175 mg/mL, 50 mg/mL to about 200 mg/mL, 50 mg/mL to about 225 mg/mL, 50 mg/mL to about 250 mg/mL, 50 mg/mL to about 275 mg/mL, 50 mg/mL to about 300 mg/mL, 50 mg/mL to about 325 mg/mL, 50 mg/mL to about 350 mg/mL, 50 mg/mL to about 375 mg/mL, 50 mg/mL to about 400 mg/mL, 50 mg/mL to about 425 mg/mL, 50 mg/mL to about 450 mg/mL, 50 mg/mL to about 475 mg/mL; 100 mg/mL to about 125 mg/mL, 100 mg/mL to about 150 mg/mL, 100 mg/mL to about 175 mg/mL, 100 mg/mL to about 200 mg/mL, 100 mg/mL to about 225 mg/mL, 100 mg/mL to about 250 mg/mL, 100 mg/mL to about 275 mg/mL, 100 mg/mL to about 300 mg/mL, 100 mg/mL to about 325 mg/mL, 100 mg/mL to about 350 mg/mL, 100 mg/mL to about 375 mg/mL, 100 mg/mL to about 400 mg/mL, 100 mg/mL to about 425 mg/mL, 100 mg/mL to about 450 mg/mL, 100 mg/mL to about 475 mg/mL, and 100 mg/mL to about 500 mg/mL. Thus, the active agent may be present in the formulation disclosed herein in an amount of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg/mL, including increments therein.

Penetration Enhancer

As noted above, the formulations described herein include a penetration enhancer. In some embodiments, the penetration enhancer is selected from diethylene glycol monoethyl ether, lauryl alcohol, dimethyl sulfoxide (DMSO), dimethyl acetamide, N-methyl pyrrolidone, oleic acid, azone, oxazolidinone derivatives, urea, terpenes (including, but not limited to, menthol, linalyl alcohol, eugenol, limonene, pinene, and squalene), or any combination thereof.

In some embodiments, the penetration enhancer comprises, consists essentially of, or consists of diethylene glycol monoethyl ether.

In some embodiments, the penetration enhancer is present in the formulation disclosed herein in an amount of about 3% w/w to about 30% w/w. This includes about 3% w/w to about 25% w/w, about 3% w/w to about 20% w/w, about 5% w/w to about 30% w/w, about 5% w/w to about 25% w/w, about 5% w/w to about 20% w/w, about 8% w/w to about 30% w/w, about 8% w/w to about 25% w/w, about 8% w/w to about 20% w/w, about 10% w/w to about 30% w/w, about 10% w/w to about 25% w/w, about 10% w/w to about 20% w/w, about 15% w/w to about 30% w/w, about 15% w/w to about 25% w/w, and about 15% w/w to about 20% w/w. In some embodiments, the penetration enhancer is present in the formulation disclosed herein in an amount of about 3, 4, 5, 6, 7, 8, 9, 10, 11, 12, 13, 14, 15, 16, 17, 18, 19, 20, 21, 22, 23, 24, 25, 26, 27, 28, 29, or 30% w/w, including increments therein. In some embodiments, the penetration enhancer is present in the formulation disclosed herein in an amount of about 18% w/w.

Thickening Agent

As noted above, the formulations described herein include a thickening agent. In some embodiments, the thickening agent is selected from a cross-linked polyacrylic acid polymer (e.g, a carbomer); a cellulose derivative (e.g., hydroxyethylcellulose, ethyl cellulose, hydroxypropyl cellulose, carboxymethyl cellulose, sodium carboxymethyl cellulose); xanthan gum, locust beam gum, guar gum or derivative thereof; alginic acid; inorganic polymer (such as Weegum, a silicate of aluminum and magnesium); PEMULEN™ (a copolymer of acrylic acid and C10-C30 alkyl acrylate cross-linked with allyl pentaerythritol); or any combination thereof.

In some embodiments, the thickening agent comprises, consists essentially of, or consists of a cross-linked polyacrylic acid polymer. In some embodiments, the cross-linked polyacrylic acid polymer is a carbomer. Commercial carbomers include, but are not limited to, CARBOPOL® polymers such as CARBOPOL® Ultrez 10 NF, CARBOPOL® Ultrez 20, CARBOPOL® ETD 2020 NF, CARBOPOL® 71G NF, CARBOPOL® 971P NF, CARBOPOL® 974P NF, CARBOPOL® 980 NF, CARBOPOL® 981 NF, and CARBOPOL® 5984 EP. CARBOPOL® Ultrez 10 NF and CARBOPOL® ETD 2020 NF are carbomer homopolymers or copolymers that contain a block copolymer of polyethylene glycol and a long chain alkyl acid ester.

In some embodiments, the thickening agent is present in the formulation disclosed herein in an amount of about 0.8% w/w to about 1.3% w/w. This includes about 0.8% w/w to about 1.2% w/w, about 0.8% w/w to about 1.1% w/w, about 0.8% w/w to about 1.0% w/w, about 0.9% w/w to about 1.3% w/w, about 0.9% w/w to about 1.2% w/w, about 0.9% w/w to about 1.1% w/w, about 1.0% w/w to about 1.3% w/w, and about 1.0% w/w to about 1.2% w/w. In some embodiments, the thickening agent is present in the formulation disclosed herein in an amount of about 0.8, 0.9, 1.0, 1.1, 1.2, or 1.3% w/w, including increments therein. In some embodiments, the thickening agent is present in the formulation disclosed herein in an amount of about 1% w/w.

Buffering Agent

As noted above, the formulations described herein include a buffering agent. In some embodiments, the buffering agent is selected from triethanolamine, sodium hydroxide, potassium hydroxide, cocoamidodiethylamine, or any combination thereof.

In some embodiments, the buffering agent comprises, consists essentially of, or consists of triethanolamine.

In some embodiments, the buffering agent is present in the formulation disclosed herein in an amount of about 0.25% w/w to about 6% w/w. This includes about 0.25% w/w to about 5% w/w, about 0.25% w/w to about 4% w/w, about 0.25% w/w to about 3% w/w, about 0.25% w/w to about 2% w/w, about 0.25% w/w to about 1% w/w, about 0.5% w/w to about 6% w/w, 0.5% w/w to about 5% w/w, about 0.5% w/w to about 4% w/w, about 0.5% w/w to about 3% w/w, about 0.5% w/w to about 2% w/w, and about 0.5% w/w to about 1% w/w. In some embodiments, the buffering agent is present in the formulation disclosed herein in an amount of about 0.25, 0.30, 0.35, 0.40, 0.45, 0.50, 0.55, 0.60, 0.65, 0.70, 0.75, 0.80, 0.85, 0.90, 0.95, 1.00, 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, or 6.00% w/w, including increments therein.

Sequestering Agent

As noted above, the formulations described herein include a sequestering agent. In some embodiments, the sequestering agent is selected from EDTA, or a salt and/or solvate thereof; citric acid; tartaric acid; or any combination thereof.

In some embodiments, the sequestering agent comprises, consists essentially of, or consists of EDTA, or a salt and/or solvate thereof.

In some embodiments, the sequestering agent is present in the formulation disclosed herein in an amount of about 0.05% w/w to about 0.08% w/w. This includes about 0.05% w/w to about 0.07% w/w, about 0.06% w/w to about 0.08% w/w, about 0.06% w/w to about 0.07% w/w, and about 0.07% w/w to about 0.08% w/w. In some embodiments, the sequestering agent is present in the formulation disclosed herein in an amount of about 0.05, 0.055, 0.06, 0.065, 0.07, 0.075, or 0.08% w/w, including increments therein.

Preservative

As noted above, the formulations described herein include a preservative. In some embodiments, the preservative is selected from phenoxyethanol, urea derivatives (such as, but not limited to, diazolidinyl urea and imidazolidinyl urea), ethylhexylglycerine, hydantoin, benzoic, sorbic acid, anisic acid, or any combination thereof.

In some embodiments, the preservative comprises, consists essentially of, or consists of phenoxyethanol.

In some embodiments, the preservative is present in the formulation disclosed herein in an amount of about 0.4% w/w to about 0.8% w/w. This includes about 0.4% w/w to about 0.7% w/w, about 0.4% w/w to about 0.6% w/w, about 0.4% w/w to about 0.5% w/w, about 0.5% w/w to about 0.8% w/w, about 0.5% w/w to about 0.7% w/w, about 0.5% w/w to about 0.6% w/w, about 0.6% w/w to about 0.8% w/w, about 0.6% w/w to about 0.7% w/w, and about 0.7% w/w to about 0.8% w/w. In some embodiments, the preservative is present in the formulation disclosed herein in an amount of about 0.4, 0.45, 0.5, 0.55, 0.6, 0.65, 0.7, 0.75, or 0.8% w/w, including increments therein.

Other Components

The formulations described herein may include one or more other components suitable for use in a transdermal composition. Deionized water is added to the formulation as needed (q.s.). The formulations described herein comprise, consist essentially of, or consist of up to about 95.45% w/w deionized water. This includes about 11.82% w/w to about 95.45% w/w, and about 31.82% w/w to about 95.45% w/w, and ranges in between. In some embodiments, the formulation comprises, consists essentially of, or consists of 79.5% w/w deionized water.

The formulations described herein are alcohol-free. The term “alcohol-free” as it pertains to a formulation described herein means that the formulation is formulated without methanol, ethanol, iso-propanol, n-propanol, tert-butanol, n-butanol, and other alcohols of similarly low boiling point.

Delivery Profile

In some embodiments, the formulation disclosed herein exhibits a delivery profile that includes a lag effect wherein, following four consecutive hourly applications of the formulation to skin, the amount of active agent delivered through the skin after 21 hours is greater than the amount delivered through the skin after 5 hours, as assessed in an in vitro permeation study using human cadaver skin. In some embodiments, the formulation disclosed herein exhibits a delivery profile that includes a lag effect wherein, following four consecutive hourly applications of the formulation to skin, the amount of phytocannabinoid delivered through the skin after 21 hours is greater than the amount delivered through the skin after 5 hours, as assessed in an in vitro permeation study using human cadaver skin.

In some embodiments, the formulation disclosed herein exhibits a lag effect wherein, following three consecutive hourly applications of the formulation to skin, the amount of active agent delivered through the skin after 22 hours is greater than the amount delivered through the skin after 5 hours, as assessed in an in vitro permeation study using human cadaver skin. In some embodiments, the formulation disclosed herein exhibits a lag effect wherein, following three consecutive hourly applications of the formulation to skin, the amount of phytocannabinoid delivered through the skin after 22 hours is greater than the amount delivered through the skin after 5 hours, as assessed in an in vitro permeation study using human cadaver skin.

In some embodiments, the formulation transdermally delivers active agent to skin in an amount of at least 10, 11, 12, 13, 14, 15, 16, 17, 18, 19, 20, 21, 22, or 23 μg/cm² of skin, as assessed in an in vitro retention study using human cadaver skin after 24 hours following an initial application of the formulation to the skin. In some embodiments, the formulation transdermally delivers phytocannabinoid to skin in an amount of at least 10, 11, 12, 13, 14, 15, 16, 17, 18, 19, 20, 21, 22, or 23 μg/cm² of skin, as assessed in an in vitro retention study using human cadaver skin after 24 hours following an initial application of the formulation to the skin.

Without being bound to any one particular theory, it is believed that lipophilicity of the formulation of the present technology is modulated such that active agent in the formulation can permeate into the skin, accumulate within the skin, and then be delivered through the skin, thereby exhibiting a lag effect as described herein.

Methods of Treatment

In another aspect, provided herein are methods of treating pain and/or inflammation in a subject in need thereof, the methods comprising, consisting essentially of, or consisting of topically administering to one or more regions of skin on the subject a therapeutically effective amount of a transdermal formulation disclosed herein.

In another aspect, provided herein are methods of treating pain in a subject in need thereof, the methods comprising, consisting essentially of, or consisting of topically administering to one or more regions of skin on the subject a therapeutically effective amount of a transdermal formulation disclosed herein.

In another aspect, provided herein are methods of treating inflammation in a subject in need thereof, the methods comprising, consisting essentially of, or consisting of topically administering to one or more regions of skin on the subject a therapeutically effective amount of a transdermal formulation disclosed herein.

In another aspect, provided herein are methods of treating musculoskeletal pain and/or inflammation in a subject in need thereof, the methods comprising, consisting essentially of, or consisting of topically administering to one or more regions of skin on the subject a therapeutically effective amount of a transdermal formulation disclosed herein.

In another aspect, provided herein are methods of treating musculoskeletal pain and/or inflammation in a subject in need thereof, the methods comprising, consisting essentially of, or consisting of administering to one or more regions of skin on the subject laser therapy and a transdermal formulation disclosed herein. The use of laser therapy has become more common for treating indications such as but not limited to chronic and acute pain conditions over the past several years. The use of lotions of any type are normally contraindicated during treatment as lotions can cause attenuation of the laser power, thus reducing the effectiveness of the laser treatment. Additionally, absorption of the laser energy within the lotion can cause a heating effect creating discomfort for the patient. It was unexpectedly found that laser therapy could be combined with administration of a transdermal formulation disclosed herein without attenuation of the effectiveness of laser therapy and without causing discomfort to the subject. In some embodiments, the transdermal formulation is topically administered. As used herein, “laser therapy” refers to application of laser light to one or more regions of a subject for therapeutic effect. Those skilled in the art can select suitable laser therapy parameters for use in such methods, such as but not limited to, laser light wavelength, laser light power, laser therapy dosage, and duration of treatment, based on principles known in the art.

In some embodiments, the musculoskeletal pain and/or inflammation is located at one or more of the foot, ankle, knee, hip, hand, wrist, elbow, neck, scalp, back, chest, abdomen, shoulder, arm, or leg, of the subject. In some embodiments, the musculoskeletal pain and/or inflammation is located at skeletal muscles of the subject.

In another aspect, provided herein are methods for relieving pain associated with osteoarthritis of one or more joints in a subject in need thereof, the methods comprising, consisting essentially of, or consisting of topically administering a therapeutically effective amount of a transdermal formulation disclosed herein to one or more regions of skin covering the one or more joints on the subject.

In some embodiments, the one or more joints are located at one or more of the foot, ankle, knee, hip, hand, wrist, elbow, neck, back, or shoulder of the subject.

In another aspect, provided herein are methods for treating acne, bacterial skin infection, dandruff, photoaging of the skin, or rosacea, or any combination thereof, in a subject in need thereof, the method comprising, consisting essentially of, or consisting of topically administering a therapeutically effective amount of a transdermal formulation described herein, wherein the active agent comprises or consists of an anti-acne agent.

In another aspect, provided herein are methods for treating allergic urticaria, anal itching, aphthous ulcer, atopic dermatitis, back pain, bacterial skin infection, external burn, cold sore, dermal ulcer, hemorrhoids, insect bites, minor cuts, minor skin irritation, muscle pain, muscle spasm, neuropathic pain, poison ivy, poison oak, poison sumac, postherpetic neuralgia, premature ejaculation, pruritus, scrapes, skin rash, sunburn, or urticaria, or any combination thereof, in a subject in need thereof, the method comprising, consisting essentially of, or consisting of topically administering a therapeutically effective amount of a transdermal formulation described herein, wherein the active agent comprises or consists of an anesthetic.

In another aspect, provided herein are methods for treating atopic dermatitis, bacterial vaginitis, basal cell carcinoma, cold sores, Condylomata acuminata, dandruff, dermal ulcer, dermatitis, eczema, head lice, herpes simplex, human papilloma viral infection, keratosis, lichen simplex chronicus, molluscum contagiosum, pruritus, rosacea, scabies, seborrheic dermatitis, or seborrheic keratosis, or any combination thereof, in a subject in need thereof, the method comprising, consisting essentially of, or consisting of topically administering a therapeutically effective amount of a transdermal formulation described herein, wherein the active agent comprises or consists of an anti-infective agent.

In another aspect, provided herein are methods for treating acne, bacterial vaginitis, balanoposthitis, head lice, perioral dermatitis, or rosacea, or any combination thereof, in a subject in need thereof, the method comprising, consisting essentially of, or consisting of topically administering a therapeutically effective amount of a transdermal formulation described herein, wherein the active agent comprises or consists of an anti-rosacea agent.

In another aspect, provided herein are methods for treating acne, bacterial skin infection, external burn, dandruff, impetigo, nasal carriage of Staphylococcus aureus, paronychia, perioral dermatitis, rosacea, seborrheic dermatitis, secondary cutaneous bacterial infections, or any combination thereof, in a subject in need thereof, the method comprising, consisting essentially of, or consisting of topically administering a therapeutically effective amount of a transdermal formulation described herein, wherein the active agent comprises or consists of an antibiotic.

In another aspect, provided herein are methods for treating androgenetic alopecia, balanoposthitis, beef tapeworm infection (Taenia saginata), cutaneous candidiasis, dandruff, diaper rash, impetigo, intertrigo, onychomycosis, fingernail onychomycosis, toenail onychomycosis, paronychia, seborrheic dermatitis, Tinea corporis, Tinea cruris, Tinea pedis, or Tinea versicolor, or any combination thereof, in a subject in need thereof, the method comprising, consisting essentially of, or consisting of topically administering a therapeutically effective amount of a transdermal formulation described herein, wherein the active agent comprises or consists of an antifungal agent.

In another aspect, provided herein are methods for treating pain, atopic dermatitis, dermatitis, eczema, lichen simplex chronicus, or pruritus, or any combination thereof, in a subject in need thereof, the method comprising, consisting essentially of, or consisting of topically administering a therapeutically effective amount of a transdermal formulation described herein, wherein the active agent comprises or consists of an antihistamine.

In another aspect, provided herein are methods for treating Condylomata acuminata, human papilloma viral infection, keratosis, molluscum contagiosum, mycosis fungoides, skin cancer, or warts, or any combination thereof, in a subject in need thereof, the method comprising, consisting essentially of, or consisting of topically administering a therapeutically effective amount of a transdermal formulation described herein, wherein the active agent comprises or consists of an anti-neoplastic.

In another aspect, provided herein are methods for treating acne, bacterial skin infection, eczema, facial wrinkles, human papilloma viral infection, impetigo, psoriasis, seborrheic dermatitis, skin pigmentation disorder, or vitiligo, or any combination thereof, in a subject in need thereof, the method comprising, consisting essentially of, or consisting of topically administering a therapeutically effective amount of a transdermal formulation described herein, wherein the active agent comprises or consists of an anti-psoriatic.

In another aspect, provided herein are methods for treating cold sores or herpes simplex in a subject in need thereof, the method comprising, consisting essentially of, or consisting of topically administering a therapeutically effective amount of a transdermal formulation described herein, wherein the active agent comprises or consists of an antiviral.

In another aspect, provided herein are methods for treating melasma in a subject in need thereof, the method comprising, consisting essentially of, or consisting of topically administering a therapeutically effective amount of a transdermal formulation described herein, wherein the active agent comprises or consists of a depigmenting agent.

In another aspect, provided herein are methods for treating acne, Condylomata acuminate, dandruff, human papilloma viral infection, or warts, or any combination thereof, in a subject in need thereof, the method comprising, consisting essentially of, or consisting of topically administering a therapeutically effective amount of a transdermal formulation described herein, wherein the active agent comprises or consists of an keratolytic.

In another aspect, provided herein are methods for treating pain, keratosis, or osteoarthritis, or any combination thereof, in a subject in need thereof, the method comprising, consisting essentially of, or consisting of topically administering a therapeutically effective amount of a transdermal formulation described herein, wherein the active agent comprises or consists of a non-steroidal anti-inflammatory drug.

In another aspect, provided herein are methods for treating keratosis or vitiligo, or any combination thereof, in a subject in need thereof, the method comprising, consisting essentially of, or consisting of topically administering a therapeutically effective amount of a transdermal formulation described herein, wherein the active agent comprises or consists of a photochemotherapeutic.

In another aspect, provided herein are methods for treating pain, bursitis, cold symptoms, dermatitis, osteoarthritis, pruritus, Raynaud's Syndrome, rheumatoid arthritis, or tendonitis, or any combination thereof, in a subject in need thereof, the method comprising, consisting essentially of, or consisting of topically administering a therapeutically effective amount of a transdermal formulation described herein, wherein the active agent comprises or consists of a rubefacient.

In another aspect, provided herein are methods for treating anal itching, recurrent aphthous stomatitis, atopic dermatitis, cutaneous T-cell lymphoma, dermatitis, dermatologic lesion, eczema, granuloma annulare, hemorrhoids, intertrigo, Lichen planus, Lichen sclerosus, necrobiosis lipoidica diabeticorum, plantar fibromatosis, pruritus, psoriasis, seborrheic dermatitis, skin rash, stomatitis, or urticaria, or any combination thereof, in a subject in need thereof, the method comprising, consisting essentially of, or consisting of topically administering a therapeutically effective amount of a transdermal formulation described herein, wherein the active agent comprises or consists of a steroid.

In another aspect, provided herein are methods for drying up oily skin in a subject in need thereof, the method comprising, consisting essentially of, or consisting of topically administering a therapeutically effective amount of a transdermal formulation described herein, wherein the active agent comprises or consists of an astringent.

In another aspect, provided herein are methods of cleaning a wound in a subject in need thereof, the method comprising, consisting essentially of, or consisting of topically administering a therapeutically effective amount of a transdermal formulation described herein, wherein the active agent comprises or consists of a debriding agent.

In another aspect, provided herein are methods of moisturizing skin in a subject in need thereof, the method, consisting essentially of, or consisting of comprising topically administering a therapeutically effective amount of a transdermal formulation described herein, wherein the active agent comprises or consists of an emollient.

In some embodiments, the transdermal formulation disclosed herein is administered by topical application to the region of skin on the subject without microneedle delivery.

In some embodiments, the transdermal formulation disclosed herein is administered once every hour for an initial period of two hours for a total of three applications and then subsequently administered 3-4 times per day. In some embodiments, the transdermal formulation disclosed herein is administered once every hour for an initial period of three hours for a total of four applications and then subsequently administered 3-4 times per day. In some embodiments, the transdermal formulation disclosed herein is administered once every hour for an initial period of four hours for a total of five applications and then subsequently administered 3-4 times per day.

Packaging

The formulations disclosed herein may be provided in any suitable container, such as a jar, a tube, or a container with a pump dispenser, optionally, a unit dose pump dispenser. In some embodiments, the formulation disclosed herein is provided in a container with a medical grade pump dispenser, optionally, a unit dose pump dispenser. In some embodiments, the formulation disclosed herein is provided in a container with a medical grade pump dispenser with a cooling tip, optionally, a unit dose pump dispenser.

The present invention, thus generally described, will be understood more readily by reference to the following examples, which are provided by way of illustration and are not intended to be limiting of the present invention.

### EXAMPLES

### Example 1. Formulation of Cannabidiol (Formulation A100)

| Constituents | Concentration (% w/w) |
|---|---|
| cannabidiol* | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

*microencapsulated (CEBIDIOL ™, Isodiol International Inc.), 100 mg/50 mL

### Example 2. Repeat Application Study—Permeation Assessment

General Protocol

Skin samples from suitable human donors shipped and stored frozen at −20° C. were used. The skins were removed from the freezer and allowed to equilibrate to room temperature. They were then punched with a steel punch to fit the top of the receptor cell. They were visually examined under stereoptic magnifier to confirm the absence of any skin defects. Receptor compartments were filled with the receptor fluid. Skin pieces were mounted on the receptor cells, the donor compartments were placed on top, and both compartments were clamped together. The skins were then allowed to hydrate in contact with the receptor fluid for ˜1 hour. Any cells showing leakage were replaced.

Five replicates of each formulation were tested versus controls (e.g., composition of the present invention versus marketed formulation). Each test formulation was applied at a dose of 10 mg and spread uniformly over a skin sample of 0.55 cm². The Franz cells were maintained at 35° C. and the receptor compartment was continuously stirred with a magnetic stir bar. A sample was taken from each receptor compartment at predetermined time intervals (e.g., 2, 4, 6/8 and 22/24 hour). The samples were assayed by HPLC.

In this particular study, the set of cells was divided into 4 groups of 5 Franz cells. Approximately 10 mg of Formulation A100 was applied to the skin of each donor compartment of the cells. The skin was obtained from a human male (63 years old, 158 lbs, back skin, 500 μm thickness).

Formulation A100 application: The first group of (5) cells was treated as in Example 2 with no further formulation application.

Formulation A100 application+1 repeat application: In the second group of (5) cells, after 1 hour, the remaining formulation at the surface of the skin samples was first removed and the samples were further wiped and cleaned quickly with a cotton swab. Then, another application of approximately 10 mg of formulation was applied.

Formulation A100 application+2 repeat applications: In the third group of (5) cells, after each of 1 hour and 2 hours, the remaining formulation at the surface of the skin samples was first removed and the samples were further wiped and cleaned quickly with a cotton swab, and then further applications of approximately 10 mg of formulation were re-applied.

Formulation A100 application+3 repeat applications: In the fourth group of (5) cells, after each of 1 hour, 2 hours, and 3 hours, the remaining formulation at the surface of the skin samples was first removed and the samples were further wiped and cleaned quickly with a cotton swab, and then further applications of approximately 10 mg of formulation were re-applied.

A sample was taken from each receptor compartment at predetermined times (2, 4, 6, 8, and 24 hours) in all study groups. All samples were analyzed by HPLC.

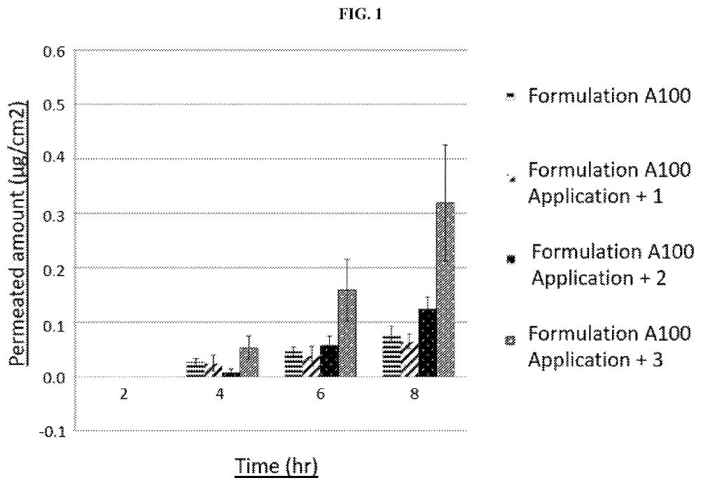

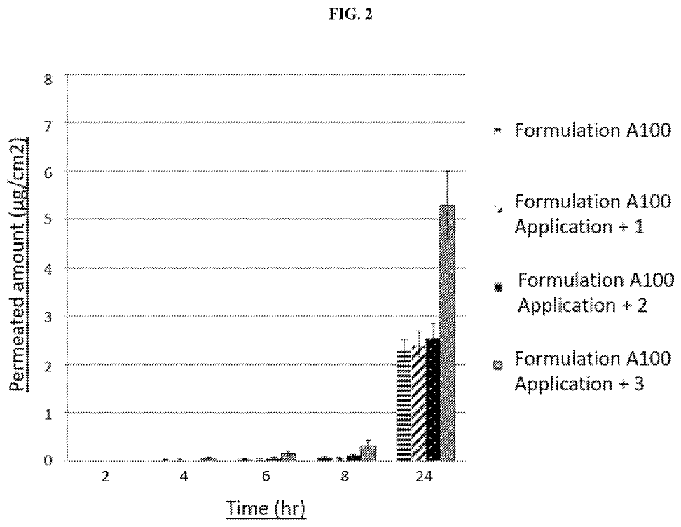

Results are shown in FIG. 1 and FIG. 2. Although administration of 3 repeat applications demonstrates the highest permeated amount after 8 hours, the permeated amount remains low (approximately 0.3 μg/cm² or less), as are the permeation amounts for the single application, 1 repeat-application and 2-repeat application (FIG. 1). Unexpectedly, the permeation values display a 16-fold to 30-fold increase after 24 hours (FIG. 2). Lag time was determined to be 3.3 hours. (Lag time is the intercept of steady-state absorption flux straight line with the time line axis which takes place after absorption has started. It reflects the delayed absorption into viable tissue related to the penetration into the stratum corneum. Direct lag time measurement in vivo is not possible but is estimated by extrapolation of the linear portion of the permeation plot to the time axis.)

### Example 3. Repeat Application Study—Retention Assessment

Skin samples were assayed to determine the amount of active agent retained in the skin as follows. After 24 hours of the permeation study of Example 2, the skin pieces were first wiped clean with cotton swabs and inspected visually to ensure that no formulation remained. The skin pieces were carefully and quickly wiped twice with Ethanol/water (80:20) impregnated cotton swabs and blotted dry each time with Kimwipes (cellulose cloths). The skins were placed into capped 1.5-dram vials to which 2 ml of ethanol (100%) were added and then sealed with Parafilm (paraffin wax). The vials were stirred overnight at 35° C. to allow the retained active agent to be extracted into ethanol. After cooling to room temperature, the samples were centrifuged and the supernatant was analyzed by HPLC.

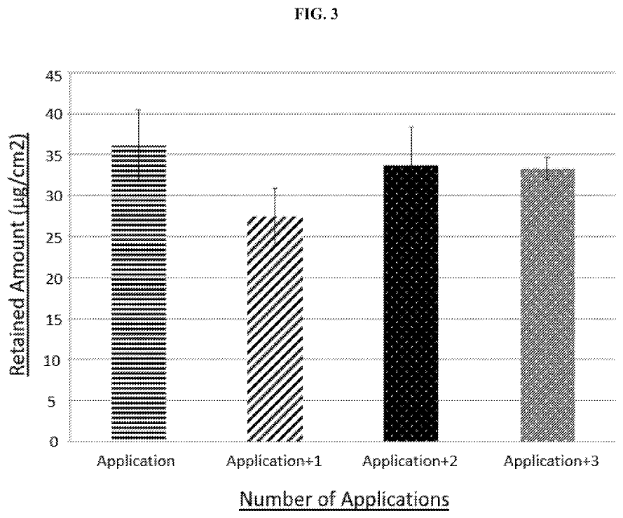

Results are shown in FIG. 3. No significant skin retention differences across the different treatment groups were observed after 24 hours.

### Example 4. Comparative Permeation Study—Formulation A100

For this study, skin pieces from a human female (66 years old, 155 lbs, back skin, 250 μm thickness), shipped and stored frozen at −20° C. were used, and prepared as described in Example 2.

Five replicates of each formulation were tested versus control (Formulation A100 versus marketed formulation). Each test formulation was applied at a dose of 10 mg and spread uniformly. The Franz cells were maintained at 35° C. and the receptor compartment was continuously stirred with a magnetic stir bar. A sample was taken from each receptor compartment at predetermined time intervals (preferably, 2, 4, 6/8 and 22/24 hour). The samples were assayed by HPLC.

The marketed formulation contained hemp extract (includes cannabidiol), camphor, menthol, beeswax, clove oil, cotton, seed oil, eucalyptus oil, jojoba seed oil, peppermint oil, sorbic acid, and tea tree oil.

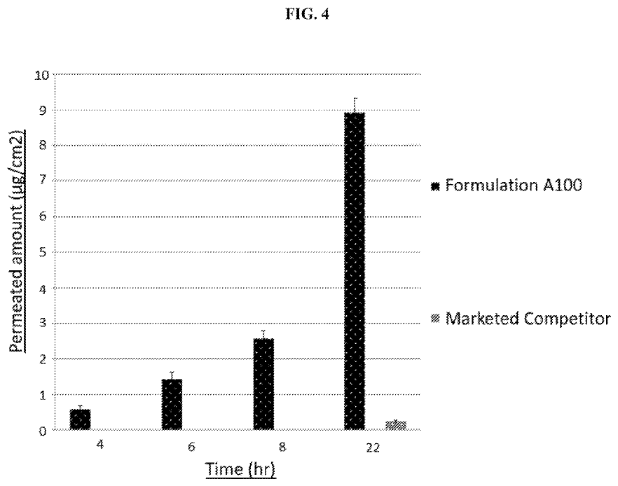

Results are shown in FIG. 4. The marketed formulation did not show any permeation as measured up to 8 hours, and showed a small amount of permeation after 22 hours.

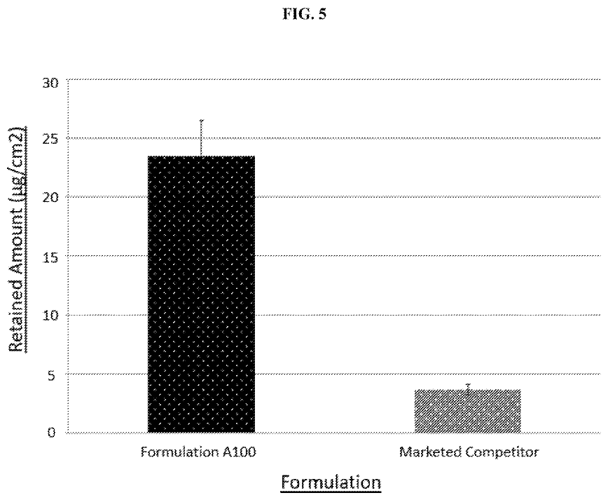

Retention was assessed as was described in Example 3. Results are shown in FIG. 5. Formulation A100 demonstrated 6-fold higher retention than the marketed formulation after 24 hours.

### Example 5. Open Label Experiential Study for Joint and/or Muscle Pain

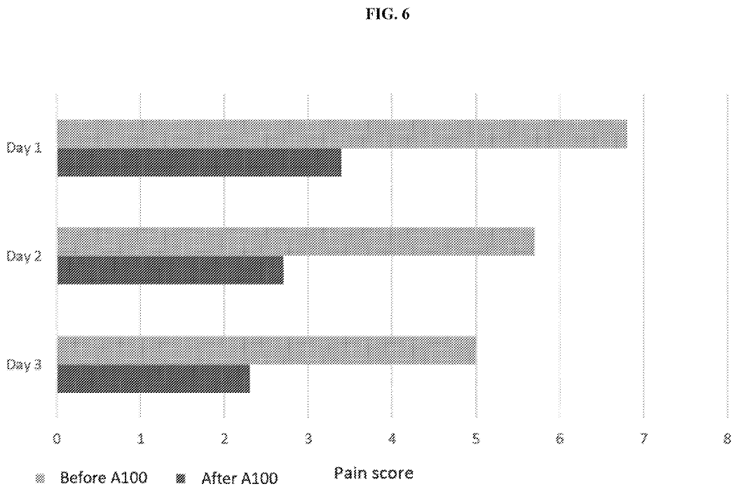

Female subjects between the age of 40-75 years old were administered Formulation A100 (formulation of Example 1) several times daily to treat joint and/or muscle pain. Each subject completed a questionnaire (designed to record the efficacy of Formulation A100 and various sensory experiences) before and after each use of Formulation A100 for 3 consecutive days. Results (n=44) regarding efficacy as recorded by the questionnaires are presented in FIG. 6. Overall pain scores decreased by 4.5 points, an improvement of 65%. Subjects reported that Formulation A100 applied smoothly and absorbed quickly into the skin without leaving a residue.

### Example 6. Formulation of Cannabigerol (Formulation A110)

An exemplary composition as described herein comprising cannabigerol as the active agent is set forth in the following table.

| Constituents | Concentration (% w/w) |
|---|---|
| cannabigerol | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 7. Formulation of Cannabichromene (Formulation A120)

An exemplary composition as described herein comprising cannabichromene as the active agent is set forth in the following table.

| Constituents | Concentration (% w/w) |
|---|---|
| cannabichromene | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 8. Formulation of Cannabinol (Formulation A130)

An exemplary composition as described herein comprising cannabinol as the active agent is set forth in the following table.

| Constituents | Concentration (% w/w) |
|---|---|
| cannabinol | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 9. Formulation of Cannabitriol (Formulation A140)

An exemplary composition as described herein comprising cannabitriol as the active agent is set forth in the following table.

| Constituents | Concentration (% w/w) |
|---|---|
| cannabitriol | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 10. Formulation of Cannabidiolic Acid (Formulation A150)

An exemplary composition as described herein comprising cannabidiolic acid as the active agent is set forth in the following table.

| Constituents | Concentration (% w/w) |
|---|---|
| cannabidiolic acid | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 11. Formulation of Cannabigerolic Acid (Formulation A160)

An exemplary composition as described herein comprising cannabigerolic acid as the active agent is set forth in the following table.

| Constituents | Concentration (% w/w) |
|---|---|
| cannabigerolic acid | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 12. Formulation of Cannabidivarin (Formulation A170)

An exemplary composition as described herein comprising cannabidivarin as the active agent is set forth in the following table.

| Constituents | Concentration (% w/w) |
|---|---|
| cannabidivarin | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 13. Formulation of Beta Caryophyllene (Formulation A180)

An exemplary composition as described herein comprising beta caryophyllene as the active agent is set forth in the following table.

| Constituents | Concentration (% w/w) |
|---|---|
| beta caryophyllene | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 14. Formulation of Benzoyl Peroxide (Formulation A190)

An exemplary composition as described herein comprising benzoyl peroxide as the active agent is set forth in the following table.

| Constituents | Concentration (% w/w) |
|---|---|
| benzoyl peroxide | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 15. Formulation of Tretinoin (Formulation A200)

An exemplary composition as described herein comprising tretinoin as the active agent is set forth in the following table.

| Constituents | Concentration (% w/w) |
|---|---|
| tretinoin | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 16. Formulation of Adapalene (Formulation A210)

An exemplary composition as described herein comprising adapalene as the active agent is set forth in the following table.

| Constituents | Concentration (% w/w) |
|---|---|
| adapalene | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 17. Formulation of Capsaicin (Formulation A220)

An exemplary composition as described herein comprising capsaicin as the active agent is set forth in the following table.

| Constituents | Concentration (% w/w) |
|---|---|
| capsaicin | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 18. Formulation of Lidocaine/Prilocaine (Formulation A230)

An exemplary composition as described herein comprising lidocaine/prilocaine as the active agent is set forth in the following table.

| Constituents | Concentration (% w/w) |
|---|---|
| lidocaine/prilocaine | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 19. Formulation of Docosanol (Formulation A240)

An exemplary composition as described herein comprising docosanol as the active agent is set forth in the following table.

| Constituents | Concentration (% w/w) |
|---|---|
| docosanol | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 20. Formulation of Mupirocin (Formulation A250)

An exemplary composition as described herein comprising mupirocin as the active agent is set forth in the following table.

| Constituents | Concentration (% w/w) |
|---|---|
| mupirocin | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 21. Formulation of Bacitracin/Polymyxin b (Formulation A260)

An exemplary composition as described herein comprising bacitracin/polymyxin b as the active agent is set forth in the following table.

| Constituents | Concentration (% w/w) |
|---|---|
| bacitracin/polymyxin b | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 22. Formulation of Clotrimazole (Formulation A270)

An exemplary composition as described herein comprising clotrimazole as the active agent is set forth in the following table.

| Constituents | Concentration (% w/w) |
|---|---|
| clotrimazole | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 23. Formulation of Tolnaftate (Formulation A280)

An exemplary composition as described herein comprising tolnaftate as the active agent is set forth in the following table.

| Constituents | Concentration (% w/w) |
|---|---|
| tolnaftate | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 24. Formulation of Miconazole (Formulation A290)

An exemplary composition as described herein comprising miconazole as the active agent is set forth in the following table.

| Constituents | Concentration (% w/w) |
|---|---|
| miconazole | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 25. Formulation of Diphenhydramine (Formulation A300)

An exemplary composition as described herein comprising diphenhydramine as the active agent is set forth in the following table.

| Constituents | Concentration (% w/w) |
|---|---|
| diphenhydramine | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 26. Formulation of Fluorouracil (Formulation A310)

An exemplary composition as described herein comprising fluorouracil as the active agent is set forth in the following table.

| Constituents | Concentration (% w/w) |
|---|---|
| fluorouracil | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 27. Formulation of Tazarotene (Formulation A320)

An exemplary composition as described herein comprising tazarotene as the active agent is set forth in the following table.

| Constituents | Concentration (% w/w) |
|---|---|
| tazarotene | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 28. Formulation of Acyclovir (Formulation A330)

An exemplary composition as described herein comprising acyclovir as the active agent is set forth in the following table.

| Constituents | Concentration (% w/w) |
|---|---|
| acyclovir | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 29. Formulation of Hydroquinone (Formulation A340)

An exemplary composition as described herein comprising hydroquinone as the active agent is set forth in the following table.

| Constituents | Concentration (% w/w) |
|---|---|
| hydroquinone | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 30. Formulation of Urea (Formulation A350)

An exemplary composition as described herein comprising urea as the active agent is set forth in the following table.

| Constituents | Concentration (% w/w) |
|---|---|
| urea | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 31 Formulation of Salicylic Acid (Formulation A360)

An exemplary composition as described herein comprising salicylic acid as the active agent is set forth in the following table.

| Constituents | Concentration (% w/w) |
|---|---|
| salicylic acid | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 32. Formulation of Diclofenac (Formulation A370)

An exemplary composition as described herein comprising diclofenac as the active agent is set forth in the following table.

| Constituents | Concentration (% w/w) |
|---|---|
| diclofenac | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 33. Formulation of Indomethacin (Formulation A380)

An exemplary composition as described herein comprising indomethacin as the active agent is set forth in the following table.

| Constituents | Concentration (% w/w) |
|---|---|
| indomethacin | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 34. Formulation of Trolamine Salicylate (Formulation A390)

An exemplary composition as described herein comprising trolamine salicylate as the active agent is set forth in the following table.

| Constituents | Concentration (% w/w) |
|---|---|
| trolamine salicylate | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 35. Formulation of Methyl Salicylate (Formulation A400)

An exemplary composition as described herein comprising methyl salicylate as the active agent is set forth in the following table.

| Constituents | Concentration (% w/w) |
|---|---|
| methyl salicylate | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 36. Formulation of Fluocinolone (Formulation A410)

An exemplary composition as described herein comprising fluocinolone as the active agent is set forth in the following table.

| Constituents | Concentration (% w/w) |
|---|---|
| fluocinolone | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 37. Formulation of Hydrocortisone (Formulation A420)

An exemplary composition as described herein comprising hydrocortisone as the active agent is set forth in the following table.

| Constituents | Concentration (% w/w) |
|---|---|
| hydrocortisone | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 38. Formulation of Minoxidil (Formulation A430)

An exemplary composition as described herein comprising minoxidil as the active agent is set forth in the following table.

| Constituents | Concentration (% w/w) |
|---|---|
| minoxidil | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 39. Formulation of Cyclobenzaprine (Formulation A440)

An exemplary composition as described herein comprising cyclobenzaprine as the active agent is set forth in the following table.

| Constituents | Concentration (% w/w) |
|---|---|
| cyclobenzaprine | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 40. Formulation of Gabapentin (Formulation A450)

An exemplary composition as described herein comprising gabapentin as the active agent is set forth in the following table.

| Constituents | Concentration (% w/w) |
|---|---|
| gabapentin | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 41. Formulation of Baclofen (Formulation A460)

An exemplary composition as described herein comprising baclofen as the active agent is set forth in the following table.

| Constituents | Concentration (% w/w) |
|---|---|
| baclofen | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 42. Formulation of Colchicine (Formulation A470)

An exemplary composition as described herein comprising colchicine as the active agent is set forth in the following table.

| Constituents | Concentration (% w/w) |
|---|---|
| colchicine | 0.2 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 43. Formulation of Ibuprofen (Formulation A480)

An exemplary composition as described herein comprising ibuprofen as the active agent is set forth in the following table.

| Constituents | Concentration (% w/w) |
|---|---|
| ibuprofen | 10 |
| diethylene glycol monoethyl ether | 18 |
| cross-linked polyacrylic acid polymer | 1 |
| (CARBOPOL ® Ultrez 30) | |
| triethanolamine | 0.3 |
| disodium EDTA dihydrate | 0.05 |
| phenoxyethanol | 0.5 |
| deionized water | q.s. |

### Example 44. Comparative Permeation Study—Formulation A480

Skin samples from a single suitable human donor shipped and stored frozen at −20° C. were used. The skin was removed from the freezer, allowed to equilibrate to room temperature, and cut to ˜2 cm×2 cm pieces prior to testing. Twelve Franz diffusion cells with a 3.3 mL receiver volume and 0.55 cm² diffusional area were used. Receptor compartments were filled with the receptor fluid (water with 2% HPBCD and 0.1 wt. % NaN₃). Skin pieces were mounted on the receptor cells, the donor compartments were placed on top, and both compartments were clamped together. The skins were then allowed to hydrate in contact with the receptor fluid for ˜20 minutes. Any cells showing leakage were replaced.

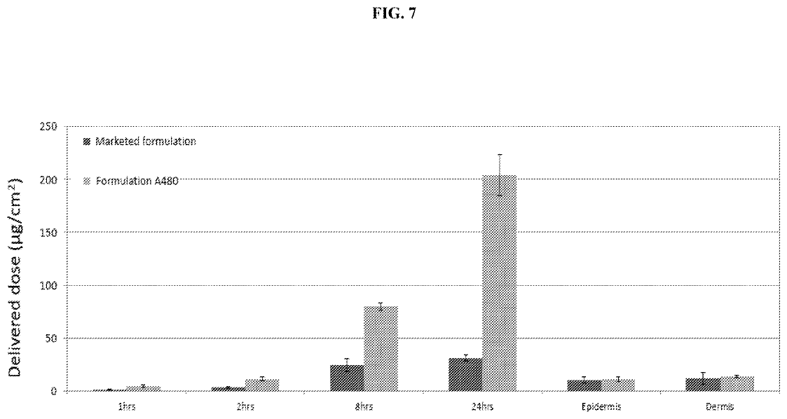

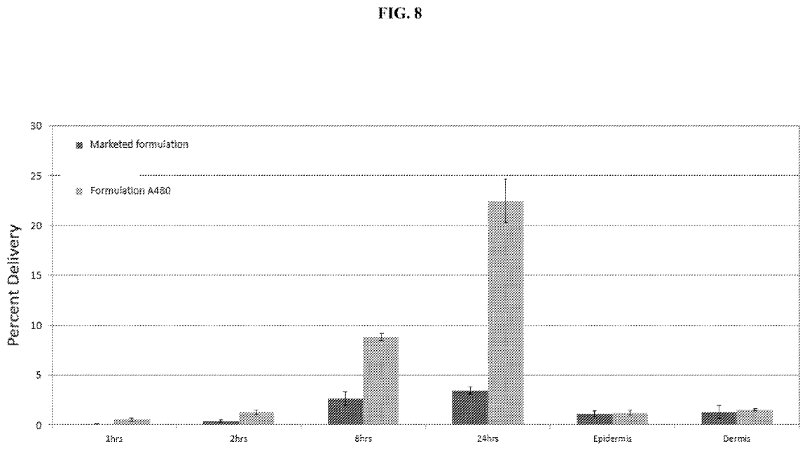

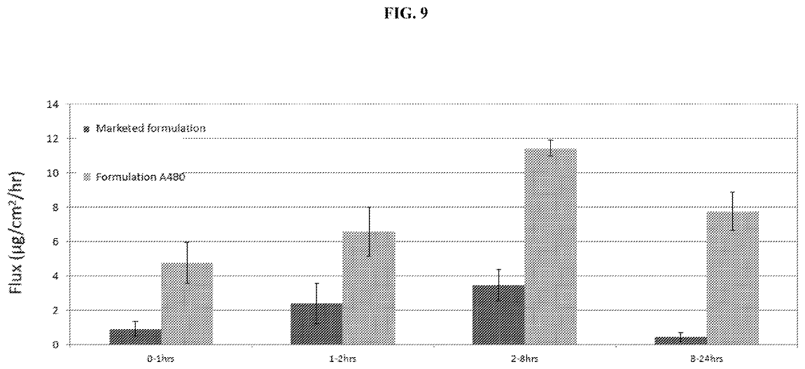

Formulation A480 was compared to a marketed formulation containing ibuprofen (10 wt. %). Each formulation was applied at a finite dose of 10 μL (corresponding to 18 mg/cm²) and spread uniformly over the addressed skin surface area. Six replicates were tested, with receptor well sampling at 4 time points (1 h, 2 h, 8 h, and 24 h). The receptor well was maintained at 32° C., and the receptor fluid in the receptor wells was stirred by magnetic stir bar throughout the experiment. Sampling was analyzed by LC-MS/MS. At the 24-hour time point, the skin pieces were washed twice with ethanol/water (1:1) and wiped dry with Kimwipes (cellulose cloths). The successive topmost layers of the stratum corneum were removed by applying cellophane tape to the skin and then pulling off the tape (3×), and the tape strips were discarded. The epidermis and dermis were separated, and extractions were performed on the epidermal and dermal skin sections. Results are shown in FIGS. 7-9.

### Example 45. Comparative Permeation Study—Formulations A110 to A470

Skin samples from a single suitable human donor shipped and stored frozen at −20° C. will be used. The skin will be removed from the freezer, allowed to equilibrate to room temperature, and cut to ˜2 cm×2 cm pieces prior to testing. Twelve Franz diffusion cells with a 3.3 mL receiver volume and 0.55 cm² diffusional area will be used. Receptor compartments will be filled with the receptor fluid (water with 2% HPBCD and 0.1 wt. % NaN₃). Skin pieces will be mounted on the receptor cells, the donor compartments will be placed on top, and both compartments will be clamped together. The skins will be then allowed to hydrate in contact with the receptor fluid for ˜20 minutes. Any cells showing leakage will be replaced.

Any one of Formulations A110 to A470 will be compared to a marketed formulation containing the corresponding active agent. Each formulation will be applied at a finite dose of 10 μL (corresponding to 18 mg/cm²) and spread uniformly over the addressed skin surface area. Six replicates will be tested, with receptor well sampling at 4 time points (1 h, 2 h, 8 h, and 24 h). The receptor well will be maintained at 32° C., and the receptor fluid in the receptor wells will be stirred by magnetic stir bar throughout the experiment. Sampling will be analyzed by LC-MS/MS. At the 24-hour time point, the skin pieces will be washed twice with ethanol/water (1:1) and wiped dry with Kimwipes (cellulose cloths). The successive topmost layers of the stratum corneum will be removed by applying cellophane tape to the skin and then pulling off the tape (3×), and the tape strips will be discarded. The epidermis and dermis will be separated, and extractions will be performed on the epidermal and dermal skin sections. It is expected that Formulations A110-A470 will have a higher delivered dose (μg/cm²) at the 8-hour and 24-hour time points than the corresponding marketed formulation.

### Example 46. Laser Absorption Characteristics of Formulation A100

An experiment was undertaken to test the percentage of attenuation of laser energy when Formulation A100 was applied.

Laser light (Epoch Laser Model 980 Therapeutic Laser) was applied to a glass slide atop a Laser Power meter measurement head at a fixed distance. The measured optical power was 4.9 watts. Formulation A100 was then applied to the clear glass slide. The laser light was then applied at the same fixed distance, and the measured optical power was 4.7 watts. The lens was then cleaned of any residue and another reading was taken and the optical power returned to 4.9 watts. The experiment was repeated a total of 2 more times for a total of three times. The resultant measurements were the same in all three instances. The percentage loss was calculated with Formulation A100 applied. The average loss was 4%.

The experiment was then repeated, this time observing the laser power reading for two minutes. There was a slight improvement after two minutes, meaning that the calculated loss decreased to 3.5% over the two-minute period and then stabilized. This also was repeated three times with similar results.

Finally, Formulation A100 was applied to the back of the experimenter's hand and the laser immediately applied thereafter using a typical therapeutic setting of 6 watts recommended by the manufacturer. No increased warming sensation was experienced when compared to the laser being applied without Formulation A100.

Conclusions. Results were consistent each time. The low percent change means that any slight reduction in power can be easily compensated for with machine setting adjustments. The results suggest that Formulation A100 does not attenuate effects of the laser when topically applied prior to laser therapy.

Para. A. A transdermal formulation comprising about 0.05% w/w to about 50% w/w of an active agent and a pharmaceutically acceptable carrier, wherein the pharmaceutically acceptable carrier comprises
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w deionized water;
   wherein the formulation does not include a phytocannabinoid.

Para. B. The formulation of Para. A, wherein the formulation is a topical formulation.

Para. C. The formulation of Para. B, wherein the topical formulation is a semi-solid formulation selected from a gel, a lotion, a cream, an ointment, a serum, or a foam.

Para. D. The formulation of any one of Paras. A-C, consisting of
about 0.20% w/w active agent,
about 18% w/w diethylene glycol monoethyl ether,
about 1% w/w cross-linked polyacrylic acid polymer,
about 0.3% w/w triethanolamine,
about 0.5% w/w phenoxyethanol,
about 0.05% w/w disodium EDTA dihydrate, and
q.s. deionized water.

Para. E. The formulation of any one of Paras. A-D, wherein the active agent is one or more selected from anti-acne agents, anesthetics, anti-infectives, anti-rosacea agents, antibiotics, antifungals, antihistamines, anti-neoplastics, anti-psoriatics, antivirals, depigmenting agents, keratolytics, non-steroidal anti-inflammatory drugs, photochemotherapeutics, rubefacients, steroids, astringents, debriding agents, and emollients.

Para. F. The formulation of Para. E, wherein the active agent comprises one or more anti-acne agents selected from benzoyl peroxide; tretinoin; adapalene; benzoyl peroxide and hydrocortisone; benzoyl peroxide and sulfur; resorcinol and sulfur; benzoyl peroxide and salicylic acid; benzoyl peroxide and erythromycin; benzoyl peroxide and clindamycin; erythromycin; benzoyl peroxide and adapalene; clindamycin and tretinoin; dapsone; salicylic acid; azelaic acid; clindamycin; and tetracycline.

Para. G. The formulation of Para. E, wherein the active agent comprises one or more anesthetics selected from capsaicin, lidocaine, menthol, and methyl salicylate; pramoxine; hydrocortisone and lidocaine; tetracaine; dibucaine; prilocaine and lidocaine; menthol and lidocaine; benzalkonium chloride and lidocaine; dyclonine; phenol; camphor, methyl salicylate, and lidocaine; capsaicin, menthol, and lidocaine; cocaine; ethyl chloride; pentafluoropropane and tetrafluoroethane; pramoxine and zinc acetate; and prilocaine and lidocaine.

Para. H. The formulation of Para. E, wherein the active agent comprises one or more anti-infectives selected from docosanol; boric acid; malathion; silver; sinecatechins; crotamiton; iodoquinol; benzyl alcohol; benzyl benzoate; cadexomer iodine; gentian violet; spinosad; ivermectin; acetic acid; imiquimod; permethrin; lindane; piperonyl butoxide and pyrethrins; hydrogen peroxide; aloe polysaccharides and iodoquinol; chloroxine; and nitrofurazone.

Para. I. The formulation of Para. E, wherein the active agent comprises one or more anti-rosacea agents selected from azelaic acid, ivermectin, metronidazole, brimonidine, and oxymetazoline.

Para. J. The formulation of Para. E, wherein the active agent comprises one or more antibiotics selected from mupirocin; bacitracin and polymyxin b; bacitracin, neomycin, polymyxin b, and pramoxine; gentamicin; sulfacetamide sodium; silver sulfadiazine; sulfur, retapamulin and sulfur; retapamulin; erythromycin; bacitracin, neomycin, and polymyxin b; pramoxine, neomycin, and polymyxin b; bacitracin; mafenide; neomycin and polymyxin b; neomycin; ozenoxacin; and tetracycline.

Para. K. The formulation of Para. E, wherein the active agent comprises one or more antifungals selected from clotrimazole; tolnaftate; miconazole; clioquinol, naftifine, miconazole and zinc oxide; oxiconazole; econazole; ciclopirox; sertaconazole; ketoconazole; undecylenic acid; nystatin; efinaconazole; terbinafine; tavaborole; butenafine; ketoconazole and pyrithione zinc; luliconazole; salicylic acid and sodium thiosulfate; and sulconazole.

Para. L. The formulation of Para. E, wherein the active agent comprises one or more antihistamines selected from diphenhydramine and doxepin.

Para. M. The formulation of Para. E, wherein the active agent comprises one or more anti-neoplastics selected from fluorouracil, imiquimod, ingenol, and mechlorethamine.

Para. N. The formulation of Para. E, wherein the active agent comprises one or more anti-psoriatics selected from tazarotene; betamethasone and calcipotriene; calcitriol; ammoniated mercury; anthralin; halobetasol and tazarotene; methoxsalen; and resorcinol.

Para. O. The formulation of Para. E, wherein the active agent comprises one or more antivirals selected from penciclovir and acyclovir.

Para. P. The formulation of Para. E, wherein the active agent comprises one or more depigmenting agents selected from fluocinolone, hydroquinone, and tretinoin; and hydroquinone.

Para. Q. The formulation of Para. E, wherein the active agent comprises one or more keratolytics selected from salicylic acid, podofilox, and Podophyllum resin.

Para. R. The formulation of Para. E, wherein the active agent comprises one or more non-steroidal anti-inflammatory drugs selected from diclofenac; indomethacin; capsaicin and diclofenac; and ibuprofen.

Para. S. The formulation of Para. E, wherein the active agent comprises one or more photochemotherapeutics selected from aminolevulini c acid, methoxsalen, and methyl aminolevulinate.

Para. T. The formulation of Para. E, wherein the active agent comprises one or more rubefacients selected from trolamine salicylate; methyl salicylate; camphor and menthol and methyl salicylate; menthol; camphor and menthol; camphor; capsaicin, menthol, and methyl salicylate; camphor and phenol; capsaicin and menthol; and menthol and methyl salicylate.

Para. U. The formulation of Para. E, wherein the active agent comprises one or more steroids selected from hydrocortisone; fluocinolone; diflorasone; prednicarbate; clocortolone; halcinonide; fluticasone; amcinonide; ammonium lactate and halobetasol; mometasone; clobetasol; flurandrenolide; desonide; betamethasone; desoximetasone; fluocinonide; halobetasol; triamcinolone; alclometasone; hydrocortisone, salicylic acid, and sulfur; and hydrocortisone and urea.

Para. V. The formulation of Para. E, wherein the active agent comprises one or more astringents selected from witch hazel; aluminum acetate; and aluminum sulfate and calcium acetate.

Para. W. The formulation of Para. E, wherein the active agent comprises one or more debriding agents selected from balsam peru, castor oil, and trypsin; and collagenase.

Para. X. The formulation of Para. E, wherein the active agent comprises one or more emollients selected from urea; aloe vera; glycerin; lanolin; salicylic acid and urea; vitamins A and D; ammonium lactate; ammonium lactate and urea; hydrocortisone and urea; lactic acid and urea; petrolatum; and vitamins A, D, and E.

Para. Y. The formulation of any one of Paras. A-D, wherein the active agent is one or more selected from cyclobenzaprine; gabapentin; baclofen; colchicine; minoxidil; balsam peru; benzoin; dexpanthenol; diphenhydramine and hydrocortisone; lactic acid; sulfur; zinc oxide; pyrithione zinc; salicylic acid and sulfur; calamine; coal tar, salicylic acid, and sulfur; aluminum chloride hexahydrate; bimatoprost; sodium hyaluronate; coal tar; eflornithine; Arnica; selenium sulfide; pimecrolimus; bentoquatam; tacrolimus; allantoin, camphor, and phenol; glycopyrronium; capsaicin; crisaborole; alitretinoi; balsam peru and castor oil; becaplermin; bexarotene; coal tar and salicylic acid; epinephrine; formaldehyde; jojoba; menthol and zinc oxide; mequinol and tretinoin; vitamin A; vitamin E; clotrimazole; dexamethasone; fluconazole; ketamine; flurbiprofen; fluticasone; or any combination thereof.

Para. Z. The formulation of any one of Paras. A-Y, wherein the formulation exhibits a lag effect wherein, following four consecutive hourly applications of the formulation to skin, the amount of the active agent delivered through the skin after 21 hours is greater than the amount delivered through the skin after 5 hours, as assessed in an in vitro permeation study using human cadaver skin.

Para. AA. The formulation of any one of Paras. A-Z, wherein the penetration enhancer comprises diethylene glycol monoethyl ether, lauryl alcohol, dimethyl sulfoxide (DMSO), dimethyl acetamide, N-methyl pyrrolidone, oleic acid, azone, oxazolidinone derivative, urea, terpene, or any combination thereof.

Para. AB. The formulation of any one of Paras. A-AA, wherein the thickening agent comprises a cross-linked polyacrylic acid polymer; a cellulose derivative; xanthan gum, locust beam gum, guar gum or derivative thereof; alginic acid; inorganic polymer; PEMULEN™ (a copolymer of acrylic acid and C10-C30 alkyl acrylate cross-linked with allyl pentaerythritol); or any combination thereof.

Para. AC. The formulation of any one of Paras. A-AB, wherein the buffering agent comprises triethanol amine, potassium hydroxide, cocoamidodiethylamine, or any combination thereof.

Para. AD. The formulation of any one of Paras. A-AC, wherein the sequestering agent comprises EDTA, or a salt and/or solvate thereof; citric acid; tartaric acid; or any combination thereof.

Para. AE. The formulation of any one of Paras. A-AD, wherein the preservative comprises phenoxyethanol, a urea derivative, ethylhexylglycerine, hydantoin, benzoic, sorbic acid, anisic acid, or any combination thereof.

Para. AF. A method for treating acne, bacterial skin infection, dandruff, photoaging of the skin, or rosacea, or any combination thereof, in a subject in need thereof, the method comprising topically administering a therapeutically effective amount of a transdermal formulation of any one of Paras. A-D and F.

Para. AG. A method for treating allergic urticaria, anal itching, aphthous ulcer, atopic dermatitis, back pain, bacterial skin infection, external burn, cold sore, dermal ulcer, hemorrhoids, insect bites, minor cuts, minor skin irritation, muscle pain, muscle spasm, neuropathic pain, poison ivy, poison oak, poison sumac, postherpetic neuralgia, premature ejaculation, pruritus, scrapes, skin rash, sunburn, or urticaria, or any combination thereof, in a subject in need thereof, the method comprising topically administering a therapeutically effective amount of a transdermal formulation of any one of Paras. A-D and G.

Para. AH. A method for treating atopic dermatitis, bacterial vaginitis, basal cell carcinoma, cold sores, Condylomata acuminata, dandruff, dermal ulcer, dermatitis, eczema, head lice, herpes simplex, human papilloma viral infection, keratosis, lichen simplex chronicus, molluscum contagiosum, pruritus, rosacea, scabies, seborrheic dermatitis, or seborrheic keratosis, or any combination thereof, in a subject in need thereof, the method comprising topically administering a therapeutically effective amount of a transdermal formulation of any one of Paras. A-D and H.

Para. AI. A method for treating acne, bacterial vaginitis, balanoposthitis, head lice, perioral dermatitis, or rosacea, or any combination thereof, in a subject in need thereof, the method comprising topically administering a therapeutically effective amount of a transdermal formulation of any one of Paras. A-D and I.

Para. AJ. A method for treating acne, bacterial skin infection, external burn, dandruff, impetigo, nasal carriage of Staphylococcus aureus, paronychia, perioral dermatitis, rosacea, seborrheic dermatitis, secondary cutaneous bacterial infections, or any combination thereof, in a subject in need thereof, the method comprising topically administering a therapeutically effective amount of a transdermal formulation of any one of Paras. A-D and J.

Para. AK. A method for treating androgenetic alopecia, balanoposthitis, beef tapeworm infection (Taenia saginata), cutaneous candidiasis, dandruff, diaper rash, impetigo, intertrigo, onychomycosis, fingernail onychomycosis, toenail onychomycosis, paronychia, seborrheic dermatitis, Tinea corporis, Tinea cruris, Tinea pedis, or Tinea versicolor, or any combination thereof, in a subject in need thereof, the method comprising topically administering a therapeutically effective amount of a transdermal formulation of any one of Paras. A-D and K.

Para. AL. A method for treating pain, atopic dermatitis, dermatitis, eczema, lichen simplex chronicus, or pruritus, or any combination thereof, in a subject in need thereof, the method comprising topically administering a therapeutically effective amount of a transdermal formulation of any one of Paras. A-D and L.

Para. AM. A method for treating Condylomata acuminata, human papilloma viral infection, keratosis, molluscum contagiosum, mycosis fungoides, skin cancer, or warts, or any combination thereof, in a subject in need thereof, the method comprising topically administering a therapeutically effective amount of a transdermal formulation of any one of Paras. A-D and M.

Para. AN. A method for treating acne, bacterial skin infection, eczema, facial wrinkles, human papilloma viral infection, impetigo, psoriasis, seborrheic dermatitis, skin pigmentation disorder, or vitiligo, or any combination thereof, in a subject in need thereof, the method comprising topically administering a therapeutically effective amount of a transdermal formulation of any one of Paras. A-D and N.

Para. AO. A method for treating cold sores or herpes simplex in a subject in need thereof, the method comprising topically administering a therapeutically effective amount of a transdermal formulation of any one of Paras. A-D and O.

Para. AP. A method for treating melasma in a subject in need thereof, the method comprising topically administering a therapeutically effective amount of a transdermal formulation of any one of Paras. A-D and P.

Para. AQ. A method for treating acne, Condylomata acuminate, dandruff, human papilloma viral infection, or warts, or any combination thereof, in a subject in need thereof, the method comprising topically administering a therapeutically effective amount of a transdermal formulation of any one of Paras. A-D and Q.

Para. AR. A method for treating pain, keratosis, or osteoarthritis, or any combination thereof, in a subject in need thereof, the method comprising topically administering a therapeutically effective amount of a transdermal formulation of any one of Paras. A-D and R.

Para. AS. A method for treating keratosis or vitiligo, or any combination thereof, in a subject in need thereof, the method comprising topically administering a therapeutically effective amount of a transdermal formulation of any one of Paras. A-D and S.

Para. AT. A method for treating pain, bursitis, cold symptoms, dermatitis, osteoarthritis, pruritus, Raynaud's Syndrome, rheumatoid arthritis, or tendonitis, or any combination thereof, in a subject in need thereof, the method comprising topically administering a therapeutically effective amount of a transdermal formulation of any one of Paras. A-D and T.

Para. AU. A method for treating anal itching, recurrent aphthous stomatitis, atopic dermatitis, cutaneous T-cell lymphoma, dermatitis, dermatologic lesion, eczema, granuloma annulare, hemorrhoids, intertrigo, Lichen planus, Lichen sclerosus, necrobiosis lipoidica diabeticorum, plantar fibromatosis, pruritus, psoriasis, seborrheic dermatitis, skin rash, stomatitis, or urticaria, or any combination thereof, in a subject in need thereof, the method comprising topically administering a therapeutically effective amount of a transdermal formulation of any one of Paras. A-D and U.

Para. AV. A method for drying up oily skin in a subject in need thereof, the method comprising topically administering a therapeutically effective amount of a transdermal formulation of any one of Paras. A-D and V.

Para. AW. A method of cleaning a wound in a subject in need thereof, the method comprising topically administering a therapeutically effective amount of a transdermal formulation of any one of Paras. A-D and W.

Para. AX. A method of moisturizing skin in a subject in need thereof, the method comprising topically administering a therapeutically effective amount of a transdermal formulation of any one of Paras. A-D and X.

Para. AY. A method of treating musculoskeletal pain and/or inflammation in a subject in need thereof, the method comprising, consisting essentially of, or consisting of administering to one or more regions of skin on the subject laser therapy and a transdermal formulation, wherein the transdermal formulation comprises, consists essentially of, or consists of about 0.05% w/w to about 50% w/w of a phytocannabinoid dispersed in a pharmaceutically acceptable carrier, wherein the pharmaceutically acceptable carrier comprises, consists essentially of, or consists of
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w deionized water.

Para. AZ. The method of Para. AY, wherein the formulation comprises, consists essentially of, or consists of:
about 0.20% w/w phytocannabinoid,
about 18% w/w diethylene glycol monoethyl ether,
about 1% w/w cross-linked polyacrylic acid polymer,
about 0.3% w/w triethanolamine,
about 0.5% w/w phenoxyethanol,
about 0.05% w/w disodium EDTA dihydrate, and
q.s deionized water.

Para. BA. The method of Para. AY or Para. AZ, wherein the phytocannabinoid is cannabidiol.

Para. BB. The method of Para. BA, wherein the cannabidiol is microencapsulated cannabidiol.

Para. BC. The method of Para. BB, wherein the microencapsulated cannabidiol comprises cannabidiol encapsulated within liposomes.

While certain embodiments have been illustrated and described, it should be understood that changes and modifications can be made therein in accordance with ordinary skill in the art without departing from the technology described herein.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. In some embodiments, “consisting essentially of” refers to the specifically recited active agent(s) as being the sole active agent(s).

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A transdermal formulation comprising about 0.05% w/w to about 50% w/w of an active agent and a pharmaceutically acceptable carrier, wherein the pharmaceutically acceptable carrier comprises
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w of deionized water;
wherein the formulation does not include a phytocannabinoid.

2. The formulation of claim 1, wherein the formulation is a topical formulation.

3. The formulation of claim 2, wherein the topical formulation is a semi-solid formulation selected from a gel, a lotion, a cream, an ointment, a serum, or a foam.

4. The formulation of claim 1, consisting of
about 0.20% w/w active agent,
about 18% w/w diethylene glycol monoethyl ether,
about 1% w/w cross-linked polyacrylic acid polymer,
about 0.3% w/w triethanolamine,
about 0.5% w/w phenoxyethanol,
about 0.05% w/w disodium EDTA dihydrate, and
q.s. deionized water.

5. The formulation of claim 1, wherein the active agent is one or more selected from anti-acne agents, anesthetics, anti-infectives, anti-rosacea agents, antibiotics, antifungals, antihistamines, anti-neoplastics, anti-psoriatics, antivirals, depigmenting agents, keratolytics, non-steroidal anti-inflammatory drugs, photochemotherapeutics, rubefacients, steroids, astringents, debriding agents, and emollients.

6. The formulation of claim 5, wherein the active agent comprises one or more anti-acne agents selected from benzoyl peroxide; tretinoin; adapalene; benzoyl peroxide and hydrocortisone; benzoyl peroxide and sulfur; resorcinol and sulfur; benzoyl peroxide and salicylic acid; benzoyl peroxide and erythromycin; benzoyl peroxide and clindamycin; erythromycin; benzoyl peroxide and adapalene; clindamycin and tretinoin; dapsone; salicylic acid; azelaic acid; clindamycin; and tetracycline.

7. (canceled)

8. The formulation of claim 5, wherein the active agent comprises one or more anti-infectives selected from docosanol; boric acid; malathion; silver; sinecatechins; crotamiton; iodoquinol; benzyl alcohol; benzyl benzoate; cadexomer iodine; gentian violet; spinosad; ivermectin; acetic acid; imiquimod; permethrin; lindane; piperonyl butoxide and pyrethrins; hydrogen peroxide; aloe polysaccharides and iodoquinol; chloroxine; and nitrofurazone.

9. (canceled)

10. (canceled)

11. The formulation of claim 5, wherein the active agent comprises one or more antifungals selected from clotrimazole; tolnaftate; miconazole; clioquinol, naftifine, miconazole and zinc oxide; oxiconazole; econazole; ciclopirox; sertaconazole; ketoconazole; undecylenic acid; nystatin; efinaconazole; terbinafine; tavaborole; butenafine; ketoconazole and pyrithione zinc; luliconazole; salicylic acid and sodium thiosulfate; and sulconazole.

12. (canceled)

13. The formulation of claim 5, wherein the active agent comprises one or more anti-neoplastics selected from fluorouracil, imiquimod, ingenol, and mechlorethamine.

14.-17. (canceled)

18. The formulation of claim 5, wherein the active agent comprises one or more non-steroidal anti-inflammatory drugs selected from diclofenac; indomethacin; capsaicin and diclofenac; and ibuprofen.

19. (canceled)

20. The formulation of claim 5, wherein the active agent comprises one or more rubefacients selected from trolamine salicylate; methyl salicylate; camphor and menthol and methyl salicylate; menthol; camphor and menthol; camphor; capsaicin, menthol, and methyl salicylate; camphor and phenol; capsaicin and menthol; and menthol and methyl salicylate.

21. The formulation of claim 5, wherein the active agent comprises one or more steroids selected from hydrocortisone; fluocinolone; diflorasone; prednicarbate; clocortolone; halcinonide; fluticasone; amcinonide; ammonium lactate and halobetasol; mometasone; clobetasol; flurandrenolide; desonide; betamethasone; desoximetasone; fluocinonide; halobetasol; triamcinolone; alclometasone; hydrocortisone, salicylic acid, and sulfur; and hydrocortisone and urea.

22.-24. (canceled)

25. The formulation of claim 1, wherein the active agent is one or more selected from cyclobenzaprine; gabapentin; baclofen; colchicine; minoxidil; balsam peru; benzoin; dexpanthenol; diphenhydramine and hydrocortisone; lactic acid; sulfur; zinc oxide; pyrithione zinc; salicylic acid and sulfur; calamine; coal tar, salicylic acid, and sulfur; aluminum chloride hexahydrate; bimatoprost; sodium hyaluronate; coal tar; eflornithine; Arnica; selenium sulfide; pimecrolimus; bentoquatam; tacrolimus; allantoin, camphor, and phenol; glycopyrronium; capsaicin; crisaborole; alitretinoi; balsam peru and castor oil; becaplermin; bexarotene; coal tar and salicylic acid; epinephrine; formaldehyde; jojoba; menthol and zinc oxide; mequinol and tretinoin; vitamin A; vitamin E; clotrimazole; dexamethasone; fluconazole; ketamine; flurbiprofen; fluticasone; and any combination thereof.

26. The formulation of claim 1, wherein the formulation exhibits a lag effect wherein, following four consecutive hourly applications of the formulation to skin, the amount of the active agent delivered through the skin after 21 hours is greater than the amount delivered through the skin after 5 hours, as assessed in an in vitro permeation study using human cadaver skin.

27. The formulation of claim 1, wherein the penetration enhancer comprises one or more selected from diethylene glycol monoethyl ether, lauryl alcohol, dimethyl sulfoxide (DMSO), dimethyl acetamide, N-methyl pyrrolidone, oleic acid, azone, oxazolidinone derivative, urea, terpene, and any combination thereof.

28. The formulation of claim 1, wherein the thickening agent comprises one or more selected from a cross-linked polyacrylic acid polymer; a cellulose derivative; xanthan gum, locust beam gum, guar gum or derivative thereof; alginic acid; inorganic polymer; PEMULEN™ (a copolymer of acrylic acid and C10-C30 alkyl acrylate cross-linked with allyl pentaerythritol); and any combination thereof.

29. The formulation of claim 1, wherein the buffering agent comprises one or more selected from triethanolamine, potassium hydroxide, cocoamidodiethylamine, and any combination thereof.

30. The formulation of claim 1, wherein the sequestering agent comprises one or more selected from EDTA, a salt of EDTA, a solvate of EDTA; citric acid; tartaric acid; and any combination thereof.

31. The formulation of claim 1, wherein the preservative comprises one or more selected from phenoxyethanol, a urea derivative, ethylhexylglycerine, hydantoin, benzoic, sorbic acid, anisic acid, and any combination thereof.

32. A method for treating one or more of acne, bacterial skin infection, dandruff, photoaging of the skin, rosacea, and any combination thereof, in a subject in need thereof, the method comprising topically administering a therapeutically effective amount of a transdermal formulation of claim 1.

33. (canceled)

34. A method for treating one or more of atopic dermatitis, bacterial vaginitis, basal cell carcinoma, cold sores, Condylomata acuminata, dandruff, dermal ulcer, dermatitis, eczema, head lice, herpes simplex, human papilloma viral infection, keratosis, lichen simplex chronicus, molluscum contagiosum, pruritus, rosacea, scabies, seborrheic dermatitis, seborrheic keratosis, and any combination thereof, in a subject in need thereof, the method comprising topically administering a therapeutically effective amount of a transdermal formulation of claim 1.

35. (canceled)

36. (canceled)

37. A method for treating one or more of androgenetic alopecia, balanoposthitis, beef tapeworm infection (Taenia saginata), cutaneous candidiasis, dandruff, diaper rash, impetigo, intertrigo, onychomycosis, fingernail onychomycosis, toenail onychomycosis, paronychia, seborrheic dermatitis, Tinea corporis, Tinea cruris, Tinea pedis, Tinea versicolor, and any combination thereof, in a subject in need thereof, the method comprising topically administering a therapeutically effective amount of a transdermal formulation of claim 1.

38. (canceled)

39. A method for treating one or more of Condylomata acuminata, human papilloma viral infection, keratosis, molluscum contagiosum, mycosis fungoides, skin cancer, warts, and any combination thereof, in a subject in need thereof, the method comprising topically administering a therapeutically effective amount of a transdermal formulation of claim 1.

40.-43. (canceled)

44. A method for treating one or more of pain, keratosis, osteoarthritis, and any combination thereof, in a subject in need thereof, the method comprising topically administering a therapeutically effective amount of a transdermal formulation of claim 1.

45. (canceled)

46. A method for treating one or more of pain, bursitis, cold symptoms, dermatitis, osteoarthritis, pruritus, Raynaud's Syndrome, rheumatoid arthritis, tendonitis, and any combination thereof, in a subject in need thereof, the method comprising topically administering a therapeutically effective amount of a transdermal formulation of claim 1.

47. A method for treating one or more of anal itching, recurrent aphthous stomatitis, atopic dermatitis, cutaneous T-cell lymphoma, dermatitis, dermatologic lesion, eczema, granuloma annulare, hemorrhoids, intertrigo, Lichen planus, Lichen sclerosus, necrobiosis lipoidica diabeticorum, plantar fibromatosis, pruritus, psoriasis, seborrheic dermatitis, skin rash, stomatitis, urticaria, and any combination thereof, in a subject in need thereof, the method comprising topically administering a therapeutically effective amount of a transdermal formulation of claim 1.

48.-50. (canceled)

51. A method of treating one or more of musculoskeletal pain and inflammation in a subject in need thereof, the method comprising administering to one or more regions of skin on the subject laser therapy and a transdermal formulation, wherein the transdermal formulation comprises about 0.05% w/w to about 50% w/w of a phytocannabinoid dispersed in a pharmaceutically acceptable carrier, wherein the pharmaceutically acceptable carrier comprises
about 3% w/w to about 30% w/w penetration enhancer,
about 0.8% w/w to about 1.3% w/w thickening agent,
about 0.25% w/w to about 6% w/w buffering agent,
about 0.05% w/w to about 0.08% w/w sequestering agent,
about 0.4% w/w to about 0.8% w/w preservative, and
up to about 95.45% w/w deionized water.
